(19) United States
(12) Patent Application Publication
(10) Pub. No.: US 2010/0056600 A1
(43) Pub. Date: Mar. 4, 2010

(54) 11BETA-HSD1 ACTIVE COMPOUNDS

(76) Inventors: Soren Ebdrup, Roskilde (DK); Janne Ejrnaes Tonder, Vaerlose (DK); Henrik Sune Andersen, Lyngby (DK)

Correspondence Address: High Point Pharmaceuticals, Inc., 4170 Mendenhall Oaks Pkwy, High Point, NC, 27265, US

(21) Appl. No.: 12/593,456

(22) PCT Filed: Mar. 27, 2008
(86) PCT No.: PCT/US08/58432

§ 371 Date: Sep. 28, 2009

Related U.S. Application Data
(60) Provisional application No. 60/920,433, filed on Mar. 28, 2007.

(51) Int. Cl. A61K 31/403; C07D 209/02; C07D 403/00; C07D 209/82; C07D 307/91; A61K 31/343; A61P 3/00; A61P 3/04; A61P 9/12

(52) U.S. Cl. 514/411; 548/452; 548/441; 549/461; 514/412; 514/468

(57) ABSTRACT

A novel class of compounds of the general formula (I), their use in therapy, pharmaceutical compositions comprising the compounds, as well as their use in the manufacture of medicaments are described. The present compounds modulate the activity of 11β-hydroxysteroid dehydrogenase type 1 (11βHSD1) and are accordingly useful in the treatment of diseases in which such a modulation is beneficial, e.g. the metabolic syndrome.

### FIELD OF INVENTION

The present invention relates to novel tricyclic compounds, to their use in therapy, to pharmaceutical compositions comprising the compounds, to the use of said compounds in the manufacture of medicaments, and to therapeutic methods comprising the administration of said compounds. The present compounds modulate the activity of 11β-hydroxysteroid dehydrogenase type 1 (11βHSD1) and are accordingly useful in the treatment of diseases in which such a modulation is beneficial, such as the metabolic syndrome.

### BACKGROUND OF THE INVENTION

The metabolic syndrome is a major global health problem. In the US, the prevalence in the adult population is currently estimated to be approximately 25%, and it continues to increase both in the US and worldwide. The metabolic syndrome is characterized by a combination of insulin resistance, dyslipidemia, obesity and hypertension leading to increased morbidity and mortality of cardiovascular diseases. People with the metabolic syndrome are at increased risk of developing frank type 2 diabetes, the prevalence of which is equally escalating.

In type 2 diabetes, obesity and dyslipidemia are also highly prevalent and around 70% of people with type 2 diabetes additionally have hypertension once again leading to increased mortality of cardiovascular diseases.

In the clinical setting, it has long been known that glucocorticoids are able to induce all of the cardinal features of the metabolic syndrome and type 2 diabetes.

11β-hydroxysteroid dehydrogenase type 1 (11βHSD1) catalyses the local generation of active glucocorticoid in several tissues and organs including predominantly the liver and adipose tissue, but also e.g. skeletal muscle, bone, pancreas, endothelium, ocular tissue and certain parts of the central nervous system. Thus, 11βHSD1 serves as a local regulator of glucocorticoid actions in the tissues and organs where it is expressed (Tannin et al., J. Biol. Chem., 266, 16653 (1991); Bujalska et al., Endocrinology, 140, 3188 (1999); Whorwood et al., J. Clin. Endocrinol. Metab., 86, 2296 (2001); Cooper et al., Bone, 27, 375 (2000); Davani et al., J. Biol. Chem., 275, 34841 (2000); Brem et al., Hypertension, 31, 459 (1998); Rauz et al., Invest. Opthalmol. Vis. Sci., 42, 2037 (2001); Moisan et al., Endocrinology, 127, 1450 (1990)).

The role of 11βHSD1 in the metabolic syndrome and type 2 diabetes is supported by several lines of evidence. In humans, treatment with the non-specific 11βHSD1 inhibitor carbenoxolone improves insulin sensitivity in lean healthy volunteers and people with type 2 diabetes. Likewise, 11βHSD1 knock-out mice are resistant to insulin resistance induced by obesity and stress. Additionally, the knock-out mice present with an anti-atherogenic lipid profile of decreased VLDL triglycerides and increased HDL-cholesterol. Conversely, mice that overexpress 11βHSD1 in adipocytes develop insulin resistance, hyperlipidemia and visceral obesity, a phenotype that resembles the human metabolic syndrome (Andrews et al., J. Clin. Endocrinol. Metab., 88, 285 (2003); Walker et al., J. Clin. Endocrinol. Metab., 80, 3155 (1995); Morton et al., J. Biol. Chem. 276, 41293 (2001); Kotelevtsev et al., Proc. Natl. Acad. Sci. USA, 94, 14924 (1997); Masuzaki et al., Science, 294, 2166 (2001)).

The more mechanistic aspects of 11βHSD1 modulation and thereby modulation of intracellular levels of active glucocorticoid have been investigated in several rodent models and different cellular systems. 11βHSD1 promotes the features of the metabolic syndrome by increasing hepatic expression of the rate-limiting enzymes in gluconeogenesis, namely phosphoenolpyuvate carboxykinase and glucose-6-phosphatase, promoting the differentiation of preadipocytes into adipocytes thus facilitating obesity, directly and indirectly stimulating hepatic VLDL secretion, decreasing hepatic LDL uptake and increasing vessel contractility (Kotelevtsev et al., Proc. Natl. Acad. Sci. USA, 94, 14924 (1997); Morton et al., J. Biol. Chem. 276, 41293 (2001); Bujalska et al., Endocrinology, 140, 3188 (1999); Souness et al., Steroids, 67, 195 (2002); Brindley & Salter, Prog. Lipid Res., 30, 349 (1991)).

WO 01/90090, WO 01/90091, WO 01/90092, WO 01/90093 and WO 01/90094 discloses various thiazol-sulfonamides as inhibitors of the human 11β-hydroxysteroid dehydrogenase type 1 enzyme, and further states that said compounds may be useful in treating diabetes, obesity, glaucoma, osteoporosis, cognitive disorders, immune disorders and depression. WO 04/089470 discloses various substituted amides as modulators of the human 11β-hydroxysteroid dehydrogenase type 1 enzyme, and further states that said compounds may be useful in treating medical disorders where a decreased intracellular concentration of active glucocorticoid is desirable. WO 2004/089415 and WO 2004/089416 discloses various combination therapies using an 11β-hydroxysteroid dehydrogenase type 1 inhibitor and respectively a glucocorticoid receptor agonist or an antihypertensive agent.

We have now found novel tricyclic compounds that modulate the activity of 11βHSD1 leading to altered intracellular concentrations of active glucocorticoid. More specifically, the present compounds inhibit the activity of 11βHSD1 leading to decreased intracellular concentrations of active glucocorticoid. Thus, the present compounds can be used to treat disorders where a decreased level of active intracellular glucocorticoid is desirable, such as e.g. the metabolic syndrome, type 2 diabetes, impaired glucose tolerance (IGT), impaired fasting glucose (IFG), dyslipidemia, obesity, hypertension, diabetic late complications, cardiovascular diseases, arteriosclerosis, atherosclerosis, myopathy, muscle wasting, osteoporosis, neurodegenerative and psychiatric disorders, and adverse effects of treatment or therapy with glucocorticoid receptor agonists.

Objects of the present invention are to provide compounds, pharmaceutical compositions and use of said compounds that modulate the activity of 11βHSD1.

### DEFINITIONS

In the following structural formulas and throughout the present specification, the following terms have the indicated meaning:

The term “monovalent radical” shall mean a chemical group attached via a single bond.

The term “monovalent radical” shall mean

The term “halogen” or “halo” means fluorine, chlorine, bromine, and iodine.

The term “hydroxy” shall mean the radical —OH.

The term “carboxy” shall mean the radical —(C═O)OH.

The term “C₁-C₄-alkyl” as used herein represents a saturated, branched or straight hydrocarbon group having the indicated number of carbon atoms, e.g. C₁₋₂-alkyl, C₁₋₃-alkyl, C₁₋₄-alkyl. Representative examples are methyl, ethyl, propyl (e.g. prop-1-yl, prop-2-yl (or iso-propyl)), butyl (e.g. 2-methylprop-2-yl (or tert-butyl), but-1-yl, but-2-yl), and the like.

The term “aryl” as used herein is intended to include monocyclic, bicyclic or polycyclic carbocyclic aromatic rings. Representative examples are phenyl, naphthyl (e.g. naphth-1-yl, naphth-2-yl), anthryl (e.g. anthr-1-yl, anthr-9-yl), phenanthryl (e.g. phenanthr-1-yl, phenanthr-9-yl), azulenyl and the like. Aryl is also intended to include monocyclic, bicyclic or polycyclic carbocyclic aromatic rings substituted with carbocyclic aromatic rings. Representative examples are biphenyl (e.g. biphenyl-2-yl, biphenyl-3-yl, biphenyl-4-yl), phenylnaphthyl (e.g. 1-phenylnaphth-2-yl, 2-phenylnaphth-1-yl), biphenylenyl and the like. Aryl is also intended to include partially saturated bicyclic or polycyclic carbocyclic rings with at least one unsaturated moiety (e.g. a benzo moiety). Representative examples are, indanyl (e.g. indan-1-yl, indan-5-yl), indenyl (e.g. inden-1-yl, inden-5-yl), 1,2,3,4-tetrahydronaphthyl (e.g. 1,2,3,4-tetrahydronaphth-1-yl, 1,2,3,4-tetrahydronaphth-2-yl, 1,2,3,4-tetrahydronaphth-6-yl), 1,2-dihydronaphthyl (e.g. 1,2-dihydronaphth-1-yl, 1,2-dihydronaphth-4-yl, 1,2-dihydronaphth-6-yl), fluorenyl (e.g. fluoren-1-yl, fluoren-4-yl, fluoren-9-yl), and the like. Aryl is also intended to include partially saturated bicyclic or polycyclic carbocyclic aromatic rings containing one or two bridges. Representative examples are, benzonorbornyl (e.g. benzonorborn-3-yl, benzonorborn-6-yl), 1,4-ethano-1,2,3,4-tetrahydronapthyl (e.g. 1,4-ethano-1,2,3,4-tetrahydronapth-2-yl, 1,4-ethano-1,2,3,4-tetrahydronapth-10-yl), and the like. Aryl is also intended to include partially saturated bicyclic or polycyclic carbocyclic aromatic rings containing one or more spiro atoms. Representative examples are spiro[cyclopentane-1,1′-indane]-4-yl, spiro[cyclopentane-1,1′-indene]-4-yl, spiro[piperidine-4,1′-indane]-1-yl, spiro[piperidine-3,2′-indane]-1-yl, spiro[piperidine-4,2′-indane]-1-yl, spiro[piperidine-4,1′-indane]-3′-yl, spiro[pyrrolidine-3,2′-indane]-1-yl, spiro[pyrrolidine-3,1′-(3′,4′-dihydronaphthalene)]-1-yl, spiro[piperidine-3,1′-(3′,4′-dihydronaphthalene)]-1-yl, spiro[piperidine-4,1′-(3′,4′-dihydronaphthalene)]-1-yl, spiro[imidazolidine-4,2′-indane]-1-yl, spiro[piperidine-4,1′-indene]-1-yl, and the like.

The term “heteroaryl” as used herein is intended to include monocyclic heterocyclic aromatic rings containing one or more heteroatoms selected from nitrogen, oxygen, sulfur, SO and S(═O)₂. Representative examples are pyrrolyl (e.g. pyrrol-1-yl, pyrrol-2-yl, pyrrol-3-yl), furanyl (e.g. furan-2-yl, furan-3-yl), thienyl (e.g. thien-2-yl, thien-3-yl), oxazolyl (e.g. oxazol-2-yl, oxazol-4-yl, oxazol-5-yl), thiazolyl (e.g. thiazol-2-yl, thiazol-4-yl, thiazol-5-yl), imidazolyl (e.g. imidazol-2-yl, imidazol-4-yl, imidazol-5-yl), pyrazolyl (e.g. pyrazol-1-yl, pyrazol-3-yl, pyrazol-5-yl), isoxazolyl (e.g. isoxazol-3-yl, isoxazol-4-yl, isoxazol-5-yl), isothiazolyl (e.g. isothiazol-3-yl, isothiazol-4-yl, isothiazol-5-yl), 1,2,3-triazolyl (e.g. 1,2,3-triazol-1-yl, 1,2,3-triazol-4-yl, 1,2,3-triazol-5-yl), 1,2,4-triazolyl (e.g. 1,2,4-triazol-1-yl, 1,2,4-triazol-3-yl, 1,2,4-triazol-5-yl), 1,2,3-oxadiazolyl (e.g. 1,2,3-oxadiazol-4-yl, 1,2,3-oxadiazol-5-yl), 1,2,4-oxadiazolyl (e.g. 1,2,4-oxadiazol-3-yl, 1,2,4-oxadiazol-5-yl), 1,2,5-oxadiazolyl (e.g. 1,2,5-oxadiazol-3-yl, 1,2,5-oxadiazol-4-yl), 1,3,4-oxadiazolyl (e.g. 1,3,4-oxadiazol-2-yl, 1,3,4-oxadiazol-5-yl), 1,2,3-thiadiazolyl (e.g. 1,2,3-thiadiazol-4-yl, 1,2,3-thiadiazol-5-yl), 1,2,4-thiadiazolyl (e.g. 1,2,4-thiadiazol-3-yl, 1,2,4-thiadiazol-5-yl), 1,2,5-thiadiazolyl (e.g. 1,2,5-thiadiazol-3-yl, 1,2,5-thiadiazol-4-yl), 1,3,4-thiadiazolyl (e.g. 1,3,4-thiadiazol-2-yl, 1,3,4-thiadiazol-5-yl), tetrazolyl (e.g. tetrazol-1-yl, tetrazol-5-yl), pyranyl (e.g. pyran-2-yl), pyridinyl (e.g. pyridine-2-yl, pyridine-3-yl, pyridine-4-yl), pyridazinyl (e.g. pyridazin-2-yl, pyridazin-3-yl), pyrimidinyl (e.g. pyrimidin-2-yl, pyrimidin-4-yl, pyrimidin-5-yl), pyrazinyl, 1,2,3-triazinyl, 1,2,4-triazinyl, 1,3,5-triazinyl, thiadiazinyl, azepinyl, azecinyl, and the like. Heteroaryl is also intended to include bicyclic heterocyclic aromatic rings containing one or more heteroatoms selected from nitrogen, oxygen, sulfur, S(═O) and S(═O)₂. Representative examples are indolyl (e.g. indol-1-yl, indol-2-yl, indol-3-yl, indol-5-yl), isoindolyl, benzofuranyl (e.g. benzo[b]furan-2-yl, benzo[b]furan-3-yl, benzo[b]furan-5-yl, benzo[c]furan-2-yl, benzo[c]furan-3-yl, benzo-[c]furan-5-yl), benzothienyl (e.g. benzo[b]thien-2-yl, benzo[b]thien-3-yl, benzo[b]thien-5-yl, benzo[c]thien-2-yl, benzo[c]thien-3-yl, benzo[c]thien-5-yl), indazolyl (e.g. indazol-1-yl, indazol-3-yl, indazol-5-yl), indolizinyl (e.g. indolizin-1-yl, indolizin-3-yl), benzopyranyl (e.g. benzo[b]pyran-3-yl, benzo[b]pyran-6-yl, benzo[c]pyran-1-yl, benzo[c]pyran-7-yl), benzimidazolyl (e.g. benzimidazol-1-yl, benzimidazol-2-yl, benzimidazol-5-yl), benzothiazolyl (e.g. benzothiazol-2-yl, benzothiazol-5-yl), benzisothiazolyl, benzoxazolyl, benzisoxazolyl, benzoxazinyl, benzotriazolyl, naphthyridinyl (e.g. 1,8-naphthyridin-2-yl, 1,7-naphthyridin-2-yl, 1,6-naphthyridin-2-yl), phthalazinyl (e.g. phthalazin-1-yl, phthalazin-5-yl), pteridinyl, purinyl (e.g. purin-2-yl, purin-6-yl, purin-7-yl, purin-8-yl, purin-9-yl), quinazolinyl (e.g. quinazolin-2-yl, quinazolin-4-yl, quinazolin-6-yl), cinnolinyl, quinoliny (e.g. quinolin-2-yl, quinolin-3-yl, quinolin-4-yl, quinolin-6-yl), isoquinolinyl (e.g. isoquinolin-1-yl, isoquinolin-3-yl, isoquinolin-4-yl), quinoxalinyl (e.g. quinoxalin-2-yl, quinoxalin-5-yl), pyrrolopyridinyl (e.g. pyrrolo[2,3-b]pyridinyl, pyrrolo[2,3-c]pyridinyl, pyrrolo[3,2-c]pyridinyl), furopyridinyl (e.g. furo[2,3-b]pyridinyl, furo[2,3-c]pyridinyl, furo[3,2-c]pyridinyl), thienopyridinyl (e.g. thieno[2,3-b]pyridinyl, thieno[2,3-c]pyridinyl, thieno[3,2-c]pyridinyl), imidazopyridinyl (e.g. imidazo[4,5-b]pyridinyl, imidazo[4,5-c]pyridinyl, imidazo[1,5-a]pyridinyl, imidazo[1,2-a]pyridinyl), imidazopyrimidinyl (e.g. imidazo[1,2-a]pyrimidinyl, imidazo[3,4-a]pyrimidinyl), pyrazolopyridinyl (e.g. pyrazolo[3,4-b]pyridinyl, pyrazolo[3,4-c]pyridinyl, pyrazolo[1,5-a]pyridinyl), pyrazolopyrimidinyl (e.g. pyrazolo[1,5-a]pyrimidinyl, pyrazolo-[3,4-d]pyrimidinyl), thiazolopyridinyl (e.g. thiazolo[3,2-d]pyridinyl), thiazolopyrimidinyl (e.g. thiazolo[5,4-d]pyrimidinyl), imdazothiazolyl (e.g. imidazo[2,1-b]thiazolyl), triazolopyridinyl (e.g. triazolo[4,5-b]pyridinyl), triazolopyrimidinyl (e.g. 8-azapurinyl), and the like. Heteroaryl is also intended to include polycyclic heterocyclic aromatic rings containing one or more heteroatoms selected from nitrogen, oxygen, sulfur, S(═O) and S(═O)₂. Representative examples are carbazolyl (e.g. carbazol-2-yl, carbazol-3-yl, carbazol-9-yl), phenoxazinyl (e.g. phenoxazin-10-yl), phenazinyl (e.g. phenazin-5-yl), acridinyl (e.g. acridin-9-yl, acridin-10-yl), phenothiazinyl (e.g. phenothiazin-10-yl), carbolinyl (e.g. pyrido[3,4-b]indol-1-yl, pyrido[3,4-b]indol-3-yl), phenanthrolinyl (e.g. phenanthrolin-5-yl), and the like. Heteroaryl is also intended to include partially saturated monocyclic, bicyclic or polycyclic heterocyclic rings containing one or more heteroatoms selected from nitrogen, oxygen, sulfur, S(═O) and S(═O)₂. Representative examples are pyrrolinyl, pyrazolinyl, imidazolinyl (e.g. 4,5-dihydroimidazol-2-yl, 4,5-dihydroimidazol-1-yl), indolinyl (e.g. 2,3-dihydroindol-1-yl, 2,3-dihydroindol-5-yl), dihydrobenzofuranyl (e.g. 2,3-dihydrobenzo[b]furan-2-yl, 2,3-dihydrobenzo[b]furan-4-yl), dihydrobenzothienyl (e.g. 2,3-dihydrobenzo[b]thien-2-yl, 2,3-dihydrobenzo[b]thien-5-yl), 4,5,6,7-tetrahydrobenzo-[b]furan-5-yl), dihydrobenzopyranyl (e.g. 3,4-dihydrobenzo[b]pyran-3-yl, 3,4-dihydrobenzo[b]pyran-6-yl, 3,4-dihydrobenzo[c]pyran-1-yl, dihydrobenzo[c]pyran-7-yl), oxazolinyl (e.g. 4,5-dihydrooxazol-2-yl, 4,5-dihydrooxazol-4-yl, 4,5-dihydrooxazol-5-yl), isoxazolinyl, oxazepinyl, tetrahydroindazolyl (e.g. 4,5,6,7-tetrahydroindazol-1-yl, 4,5,6,7-tetrahydroindazol-3-yl, 4,5,6,7-tetrahydroindazol-4-yl, 4,5,6,7-tetrahydroindazol-6-yl), tetrahydrobenzimidazolyl (e.g. 4,5,6,7-tetrahydrobenzimidazol-1-yl, 4,5,6,7-tetrahydrobenzimidazol-5-yl), tetrahydroimidazo[4,5-c]pyridyl (e.g. 4,5,6,7-tetrahydroimidazo[4,5-c]pyrid-1-yl, 4,5,6,7-tetrahydroimidazo[4,5-c]pyrid-5-yl, 4,5,6,7-tetrahydroimidazo[4,5-c]pyrid-6-yl), tetrahydroquinolinyl (e.g. 1,2,3,4-tetrahydroquinolinyl, 5,6,7,8-tetrahydroquinolinyl), tetrahydroisoquinolinyl (e.g. 1,2,3,4-tetrahydroisoquinolinyl, 5,6,7,8-tetrahydroisoquinolinyl), tetrahydroquinoxalinyl (e.g. 1,2,3,4-tetrahydroquinoxalinyl, 5,6,7,8-tetrahydroquinoxalinyl), and the like. Heteroaryl is also intended to include partially saturated bicyclic or polycyclic heterocyclic rings containing one or more spiro atoms. Representative examples are spiro[isoquinoline-3,1′-cyclohexan]-1-yl, spiro-[piperidine-4,1′-benzo[c]thiophen]-1-yl, spiro[piperidine-4,1′-benzo[c]furan]-1-yl, spiro-[piperidine-4,3′-benzo[b]furan]-1-yl, spiro[piperidine-4,3′-coumarin]-1-yl, and the like.

Certain of the above defined terms may occur more than once in the structural formulae, and upon such occurrence each term shall be defined independently of the other.

Certain of the defined terms may occur in combinations, and it is to be understood that the first mentioned radical is a substituent on the subsequently mentioned radical, where the point of substitution, i.e. the point of attachment to another part of the molecule, is on the last mentioned of the radicals.

The term “optionally substituted” as used herein means that the groups in question are either unsubstituted or substituted with one or more of the substituents specified. When the groups in question are substituted with more than one substituent the substituents may be the same or different.

The term “treatment” is defined as the management and care of a patient for the purpose of combating or alleviating the disease, condition or disorder, and the term includes the administration of the active compound to prevent the onset of the symptoms or complications, or alleviating the symptoms or complications, or eliminating the disease, condition, or disorder.

The term “pharmaceutically acceptable” is defined as being suitable for administration to humans without adverse events.

The term “prodrug” is defined as a chemically modified form of the active drug, said prodrug being administered to the patient and subsequently being converted to the active drug. Techniques for development of prodrugs are well known in the art.

### SUMMARY OF THE INVENTION

In one aspect, the present invention provides a compound of the general formula I.

The present invention furthermore relates to the use in therapy of the compounds according to the invention, to pharmaceutical compositions comprising the compounds, to the use of said compounds in the manufacture of medicaments, and to therapeutic methods comprising the administration of said compounds.

### DETAILED DESCRIPTION OF THE INVENTION

The present invention is based on the observation that the compounds of the general formulas (I) disclosed below are able to modulate or inhibit the activity of 11βHSD1.

Accordingly, the present invention is concerned with compounds or prodrugs thereof of the general formula (I):

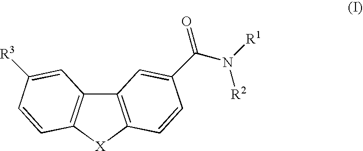

wherein

### X is CR⁴R⁵, C═O, NR⁴, O, S, or SO₂;

R¹ is hydrogen or C₁-C₄alkyl;
R² is a monovalent radical having one of the following formulae, wherein the symbol (*) denotes the point of attachment:

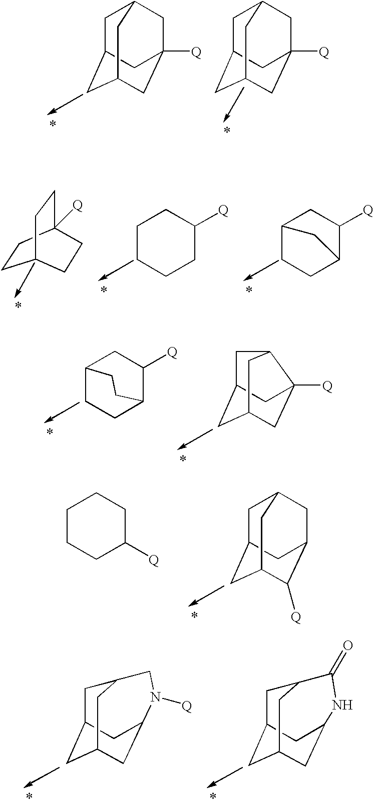

wherein Q is hydroxy, hydroxymethyl, carboxy, —C(═O)—NR⁴R⁵, —S(═O)₂NR⁴R⁵, or S(═O)₂R⁶; or
R¹ and R² together with the nitrogen to which they are attached forms one of the following formulae wherein the symbol (*) denotes the point of attachment:

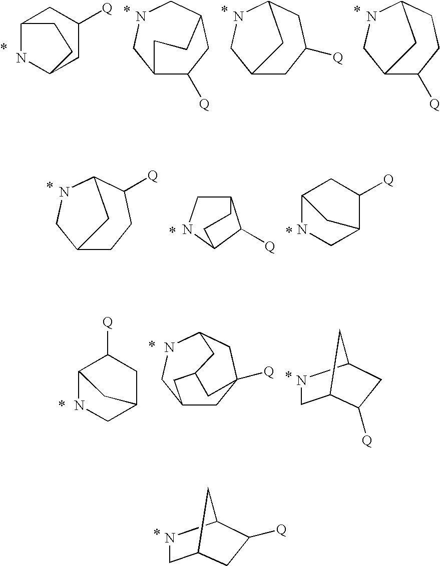

wherein Q is hydroxy, hydroxymethyl, carboxy, —C(═O)—NR⁴R⁵, —S(═O)₂NR⁴R⁵, or S(═O)₂R⁶;
R³ is hydrogen, halogen, hydroxy, cyano, C₁-C₄alkyl, aryl, heteroaryl, —NR⁴R⁵, —OR⁶, —SR⁶, or SO₂R⁶, wherein said alkyl, aryl and heteroaryl groups are optionally substituted with one or two independently selected R⁷;
R⁴ and R⁵ independently are hydrogen or C₁-C₄alkyl, wherein said C₁-C₄alkyl is optionally substituted with one or two independently selected R⁷;
R⁶ is hydrogen or C₁-C₄alky, wherein said C₁-C₄alkyl is optionally substituted with hydroxy;
R⁷ is selected from the group consisting of hydrogen, cyano, C₁-C₄alkyl, cyclopropyl, hydroxy, halogen, trifluoromethyl, —CH₂OH and carboxy; or
a salt thereof with a pharmaceutically acceptable acid or base, or any optical isomer or mixture of optical isomers, including a racemic mixture, or any tautomeric forms.

In one embodiment of the present invention, in formula (I) X is CR⁴R⁵, C═O or NR⁴, wherein R⁴ and R⁵ are as defined above.

In another embodiment of the present invention, in formula (I) X is CR⁴R⁵, wherein R⁴ and R⁵ are as defined above.

In another embodiment of the present invention, in formula (I) X is CH₂.

In another embodiment of the present invention, in formula (I) X is O.

In another embodiment of the present invention, in formula (I), X is NR⁴, wherein R⁴ is as defined above.

In another embodiment of the present invention, in formula (I), X is NH or NCH₃.

In another embodiment of the present invention, in formula (I), R¹ is hydrogen or C₁-C₄alkyl.

In another embodiment of the present invention, in formula (I), R¹ is hydrogen.

In another embodiment of the present invention, in formula (I), R¹ is C₁-C₄alkyl.

In another embodiment of the present invention, in formula (I), R¹ is methyl.

In another embodiment of the present invention, in formula (I), R¹ is ethyl.

In another embodiment of the present invention, in formula (I), Q is hydroxy, hydroxymethyl or carboxy.

In another embodiment of the present invention, in formula (I), Q is —C(═O)—NR⁴R⁵, S(═O)₂NR⁴R⁵, or S(═O)₂R⁶, wherein R⁴, R⁵ and R⁶ are as defined above.

In another embodiment of the present invention, in formula (I), Q is hydroxy.

In another embodiment of the present invention, in formula (I), R² is a monovalent radical having one of the following formulae, wherein the symbol (*) denotes the point of attachment:

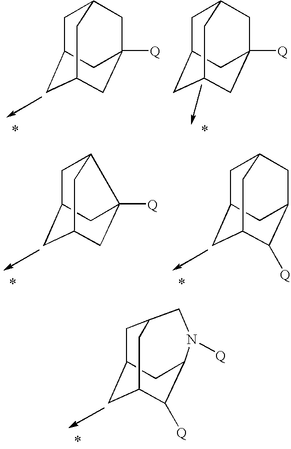

wherein Q is as defined above.

In another embodiment of the present invention, in formula (I), R² is a monovalent radical having one of the following formulae, wherein the symbol (*) denotes the point of attachment:

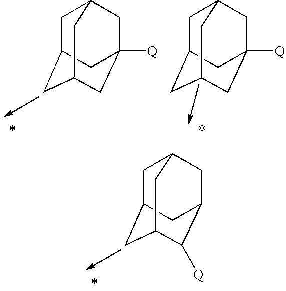

wherein Q is hydroxy.

In another embodiment of the present invention, in formula (I), R¹ and R² together with the nitrogen to which they are attached, forms together with the nitrogen to which they are attached forms one of the following formulae wherein the symbol (*) denotes the point of attachment:

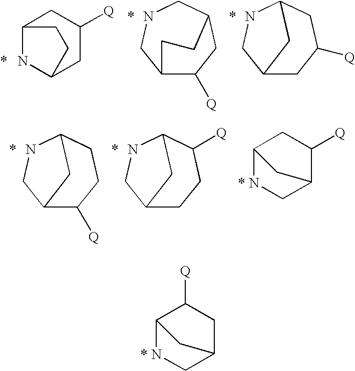

wherein Q is as defined above.

In another embodiment of the present invention, in formula (I), R¹ and R² together with the nitrogen to which they are attached, forms together with the nitrogen to which they are attached forms one of the following formulae wherein the symbol (*) denotes the point of attachment:

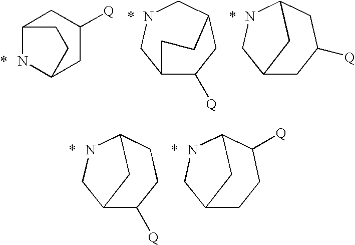

wherein Q is as defined above.

In another embodiment of the present invention, in formula (I), R¹ and R² together with the nitrogen to which they are attached, forms together with the nitrogen to which they are attached forms one of the following formulae wherein the symbol (*) denotes the point of attachment:

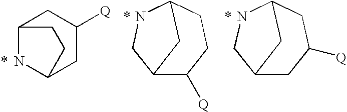

wherein Q is as defined above.

In another embodiment of the present invention, in formula (I), R¹ and R² together with the nitrogen to which they are attached, forms together with the nitrogen to which they are attached forms the following formula wherein the symbol (*) denotes the point of attachment:

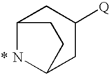

wherein Q is as defined above.

In another embodiment of the present invention, in formula (I), R¹ and R² together with the nitrogen to which they are attached, forms together with the nitrogen to which they are attached forms the following formula wherein the symbol (*) denotes the point of attachment:

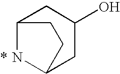

In another embodiment of the present invention, in formula (I), R³ is hydrogen, halogen, hydroxy, cyano or C₁-C₄alkyl.

In another embodiment of the present invention, in formula (I), R³ is hydrogen, halogen, hydroxy or C₁-C₄alkyl.

In another embodiment of the present invention, in formula (I), R³ is hydrogen.

In another embodiment of the present invention, the compounds of general formula (I) is selected from the group consisting of:

| Molecule | Name (IUPAC) |
|---|---|
| 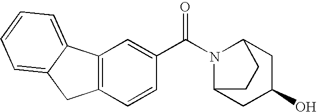 | (9H-Fluoren-3-yl)-(cis 3-hydroxy-8-aza-bicyclo- [3.2.1]oct-8-yl)-methanone |
| 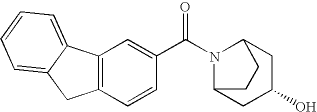 | (9H-Fluoren-3-yl)-(trans 3-hydroxy-8-aza-bicyclo- [3.2.1]oct-8-yl)-methanone |
| 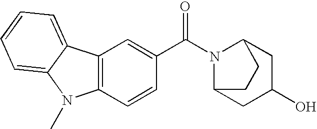 | (3-Hydroxy-8-aza-bicyclo[3.2.1]oct-8-yl)-(9-methyl-9H- carbazol-3-yl)-methanone |
| 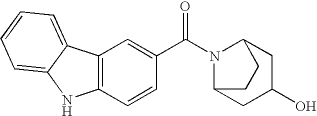 | (9H-Carbazol-3-yl)-(3-hydroxy-8-aza-bicyclo[3.2.1]oct- 8-yl)-methanone |
| 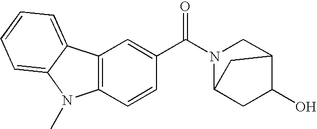 | (5-Hydroxy-2-aza-bicyclo[2.2.1]hept-2-yl)-(9-methyl-9H- carbazol-3-yl)-methanone |
| 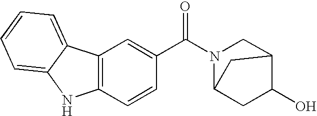 | (9H-Carbazol-3-yl)-(5-hydroxy-2-aza-bicyclo[2.2.1] hept-2-yl)-methanone |
| 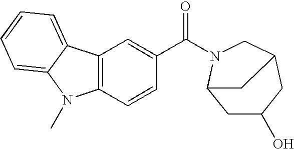 | (3-Hydroxy-6-aza-bicyclo[3.2.1]oct-6-yl)-(9-methyl-9H- carbazol-3-yl)-methanone |
| 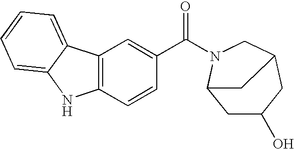 | (9H-Carbazol-3-yl)-(3-hydroxy-6-aza-bicyclo[3.2.1]oct- 6-yl)-methanone |
| 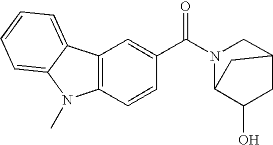 | (6-Hydroxy-2-aza-bicyclo[2.2.1]hept-2-yl)-(9-methyl- 9H-carbazol-3-yl)-methanone |
| 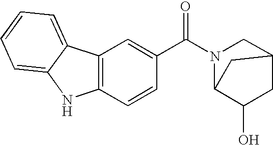 | (9H-Carbazol-3-yl)-(6-hydroxy-2-aza-bicyclo[2.2.1]- hept-2-yl)-methanone |
| 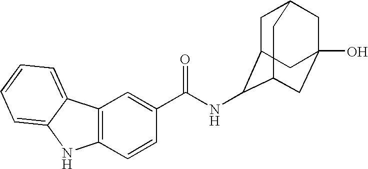 | 9H-Carbazole-3-carboxylic acid (5-hydroxy- adamantan-2-yl)-amide |
| 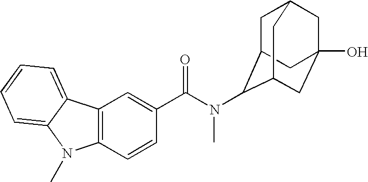 | 9-Methyl-9H-carbazole-3-carboxylic acid (5-hydroxy- adamantan-2-yl)-methyl-amide |
| 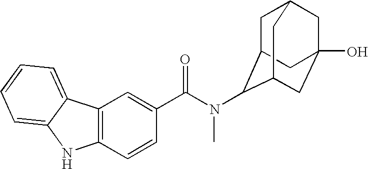 | 9H-Carbazole-3-carboxylic acid (5-hydroxy- adamantan-2-yl)-methyl-amide |
| 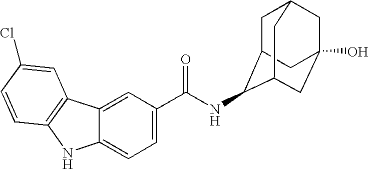 | 6-Chloro-9H-carbazole-3-carboxylic acid (trans 5- hydroxy-adamantan-2-yl)-amide |
| 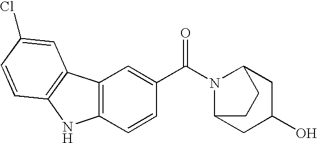 | (6-Chloro-9H-carbazol-3-yl)-(3-hydroxy-8-aza- bicyclo[3.2.1]oct-8-yl)-methanone |
| 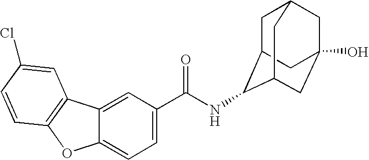 | 6-Chloro-9H-carbazole-3-carboxylic acid (cis 5- hydroxy-adamantan-2-yl)-amide |
| 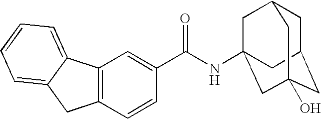 | 9H-Fluorene-3-carboxylic acid (3-hydroxy-adamantan- 1-yl)-amide |
| 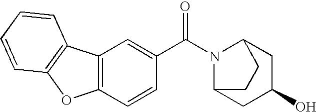 | Dibenzofuran-2-yl-(3-hydroxy-8-aza-bicyclo[3.2.1]oct- 8-yl)-methanone |
| 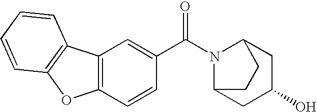 | Dibenzofuran-2-yl-(3-hydroxy-8-aza-bicyclo[3.2.1]oct- 8-yl)-methanone |
| 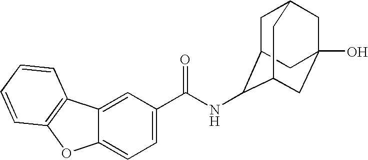 | Dibenzofuran-2-carboxylic acid (5-hydroxy- adamantan-2-yl)-amide |
| 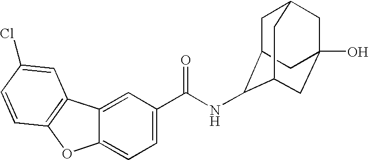 | 8-Chloro-dibenzofuran-2-carboxylic acid (5-hydroxy- adamantan-2-yl)-amide |
| 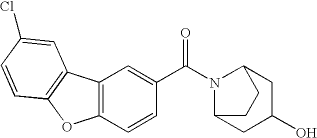 | (8-Chloro-dibenzofuran-2-yl)-(3-hydroxy-8-aza- bicyclo[3.2.1]oct-8-yl)-methanone |
| 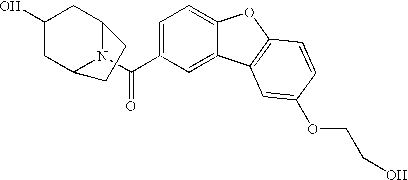 | (3-Hydroxy-8-aza-bicyclo[3.2.1]oct-8-yl)-[8-(2-hydroxy- ethoxy)-dibenzofuran-2-yl]-methanone |
| 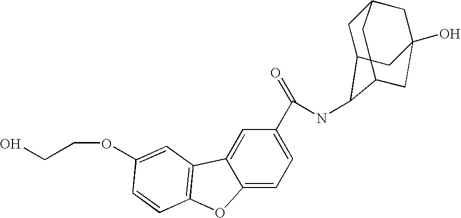 | 8-(2-Hydroxy-ethoxy)-dibenzofuran-2-carboxylic acid (5-hydroxy-adamantan-2-yl)-amide |

or a prodrug thereof, a salt thereof with a pharmaceutically acceptable acid or base, or any optical isomer or mixture of optical isomers, including a racemic mixture, or any tautomeric forms.

In one aspect of the invention, the compounds according to the invention have a IC₅₀ value as tested as described under the heading “PHARMACOLOGICAL METHODS” below 1500 nM, in a further aspect below 500 nM, in yet a further aspect below 300 nM and in yet a further aspect below 200 nM.

The compounds of the present invention have asymmetric centers and may occur as racemates, racemic mixtures, and as individual enantiomers or diastereoisomers, with all isomeric forms being included in the present invention as well as mixtures thereof.

The present invention also encompasses pharmaceutically acceptable salts of the present compounds. Such salts include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Further examples of pharmaceutically acceptable inorganic or organic acid addition salts include the pharmaceutically acceptable salts listed in J. Pharm. Sci., 66, 2 (1977), which is incorporated herein by reference. Examples of metal salts include lithium, sodium, potassium, barium, calcium, magnesium, zinc, calcium salts and the like. Examples of amines and organic amines include ammonium, methylamine, dimethylamine, trimethylamine, ethylamine, diethylamine, propylamine, butylamine, tetramethylamine, ethanolamine, diethanolamine, triethanolamine, meglumine, ethylenediamine, choline, N,N′-dibenzylethylenediamine, N-benzylphenylethylamine, N-methyl-D-glucamine, guanidine and the like. Examples of cationic amino acids include lysine, arginine, histidine and the like.

Further, some of the compounds of the present invention may form solvates with water or common organic solvents. Such solvates are encompassed within the scope of the invention.

The pharmaceutically acceptable salts are prepared by reacting a compound of the present invention with 1 to 4 equivalents of a base such as sodium hydroxide, sodium methoxide, sodium hydride, potassium tert-butoxide, calcium hydroxide, magnesium hydroxide and the like, in solvents like ether, THF, methanol, tert-butanol, dioxane, isopropanol, ethanol etc. Mixtures of solvents may be used. Organic bases like lysine, arginine, diethanolamine, choline, guandine and their derivatives etc. may also be used. Alternatively, acid addition salts wherever applicable are prepared by treatment with acids such as hydrochloric acid, hydrobromic acid, nitric acid, sulfuric acid, phosphoric acid, p-toluenesulphonic acid, methanesulfonic acid, acetic acid, citric acid, maleic acid salicylic acid, hydroxynaphthoic acid, ascorbic acid, palmitic acid, succinic acid, benzoic acid, benzenesulfonic acid, tartaric acid and the like in solvents like ethyl acetate, ether, alcohols, acetone, THF, dioxane etc. Mixture of solvents may also be used.

The stereoisomers of the compounds forming part of this invention may be prepared by using reactants in their single enantiomeric form in the process wherever possible or by conducting the reaction in the presence of reagents or catalysts in their single enantiomer form or by resolving the mixture of stereoisomers by conventional methods. Some of the preferred methods include use of microbial resolution, enzymatic resolution, resolving the diastereomeric salts formed with chiral acids such as mandelic acid, camphorsulfonic acid, tartaric acid, lactic acid, and the like wherever applicable or chiral bases such as brucine, (R)- or (S)-phenylethylamine, cinchona alkaloids and their derivatives and the like. Commonly used methods are compiled by Jaques et al. in “Enantiomers, Racemates and Resolution” (Wiley Interscience, 1981). More specifically the compound of the present invention may be converted to a 1:1 mixture of diastereomeric amides by treating with chiral amines, aminoacids, aminoalcohols derived from aminoacids; conventional reaction conditions may be employed to convert acid into an amide; the diastereomers may be separated either by fractional crystallization or chromatography and the stereoisomers of compound of formula I may be prepared by hydrolysing the pure diastereomeric amide.

Various polymorphs of the compounds forming part of this invention may be prepared by crystallization of said compounds under different conditions; for example, using different solvents commonly used or their mixtures for recrystallization; crystallizations at different temperatures; or various modes of cooling, ranging from very fast to very slow cooling during crystallizations. Polymorphs may also be obtained by heating or melting the compound followed by gradual or fast cooling. The presence of polymorphs may be determined by solid probe nmr spectroscopy, it spectroscopy, differential scanning calorimetry, powder X-ray diffraction or such other techniques.

The invention also encompasses prodrugs of the present compounds, which on administration undergo chemical conversion by metabolic processes before becoming active pharmacological substances. In general, such prodrugs will be functional derivatives of the present compounds, which are readily convertible in vivo into the required compound of the present invention. Conventional procedures for the selection and preparation of suitable prodrug derivatives are described, for example, in “Design of Prodrugs”, ed. H. Bundgaard, Elsevier, 1985.

It is a well known problem in drug discovery that compounds, such as enzyme inhibitors, may be very potent and selective in biochemical assays, yet be inactive in vivo. This lack of so-called bioavailability may be ascribed to a number of different factors such as lack of or poor absorption in the gut, first pass metabolism in the liver and/or poor uptake in cells. Although the factors determining bioavailability are not completely understood, there are many examples in the scientific literature—well known to those skilled in the art—of how to modify compounds, which are potent and selective in biochemical assays but show low or no activity in vivo, into drugs that are biologically active.

It is within the scope of the invention to modify the compounds of the present invention, termed the ‘original compound’, by attaching chemical groups that will improve the bioavailability of said compounds in such a way that the uptake in cells or mammals is facilitated.

Examples of said modifications, which are not intended in any way to limit the scope of the invention, include changing of one or more carboxy groups to esters (for instance methyl esters, ethyl esters, tert-butyl, acetoxymethyl, pivaloyloxymethyl esters or other acyloxymethyl esters). Compounds of the invention, original compounds, such modified by attaching chemical groups are termed ‘modified compounds’.

The invention also encompasses active metabolites of the present compounds.

The compounds according to the invention alter, and more specifically, reduce the level of active intracellular glucocorticoid and are accordingly useful for the treatment, prevention and/or prophylaxis of disorders and diseases in which such a modulation or reduction is beneficial.

Accordingly, the present compounds may be applicable for the treatment, prevention and/or prophylaxis of the metabolic syndrome, insulin resistance, dyslipidemia, hypertension, obesity, type 2 diabetes, impaired glucose tolerance (IGT), impaired fasting glucose (IFG), Latent Autoimmune Diabetes in the Adult (LADA), type 1 diabetes, diabetic late complications including cardiovascular diseases, cardiovascular disorders, disorders of lipid metabolism, neurodegenerative and psychiatric disorders, dysregulation of intraocular pressure including glaucoma, immune disorders, inappropriate immune responses, musculo-skeletal disorders, gastrointestinal disorders, polycystic ovarie syndrome (PCOS), reduced hair growth or other diseases, disorders or conditions that are influenced by intracellular glucocorticoid levels, adverse effects of increased blood levels of active endogenous or exogenous glucocorticoid, and any combination thereof, adverse effects of increased plasma levels of endogenous active glucocorticoid, Cushing's disease, Cushing's syndrome, adverse effects of glucocorticoid receptor agonist treatment of autoimmune diseases, adverse effects of glucocorticoid receptor agonist treatment of inflammatory diseases, adverse effects of glucocorticoid receptor agonist treatment of diseases with an inflammatory component, adverse effects of glucocorticoid receptor agonist treatment as a part of cancer chemotherapy, adverse effects of glucocorticoid receptor agonist treatment for surgical/post-surgical or other trauma, adverse effects of glucocorticoid receptor agonist therapy in the context of organ or tissue transplantation or adverse effects of glucocorticoid receptor agonist treatment in other diseases, disorders or conditions where glucocorticoid receptor agonists provide clinically beneficial effects. Also the present compounds may be applicable for the treatment of visceral fat accumulation and insulin resistance in HAART (highly active antiretroviral treatment)-treated patients. Further, the present compounds may be applicable for the treatment of hydrocephalus as well as for the treatment or prevention of disorders related to the buildup of fluid within the ventricles of the brain.

More specifically the present compounds may be applicable for the treatment, prevention and/or prophylaxis of the metabolic syndrome, type 2 diabetes, diabetes as a consequence of obesity, insulin resistance, hyperglycemia, prandial hyperglycemia, hyperinsulinemia, inappropriately low insulin secretion, impaired glucose tolerance (IGT), impaired fasting glucose (IFG), increased hepatic glucose production, type 1 diabetes, LADA, pediatric diabetes, dyslipidemia, diabetic dyslipidemia, hyperlipidemia, hypertriglyceridemia, hyperlipoproteinemia, hypercholesterolemia, decreased HDL cholesterol, impaired LDL/HDL ratio, other disorders of lipid metabolism, obesity, visceral obesity, obesity as a consequence of diabetes, increased food intake, hypertension, diabetic late complications, micro-/macroalbuminuria, nephropathy, retinopathy, neuropathy, diabetic ulcers, cardiovascular diseases, arteriosclerosis, atherosclerosis, coronary artery disease, cardiac hypertrophy, myocardial ischemia, heart insufficiency, congestional heart failure, stroke, myocardial infarction, arrythmia, decreased blood flow, erectile dysfunction (male or female), myopathy, loss of muscle tissue, muscle wasting, muscle catabolism, osteoporosis, decreased linear growth, neurodegenerative and psychiatric disorders, Alzheimers disease, neuronal death, impaired cognitive function, depression, anxiety, eating disorders, appetite regulation, migraine, epilepsia, addiction to chemical substances, disorders of intraocular pressure, glaucoma, polycystic ovary syndrome (PCOS), inappropriate immune responses, inappropriate T helper-1/T helper-2 polarisation, bacterial infections, mycobacterial infections, fungal infections, viral infections, parasitic infestations, suboptimal responses to immunizations, immune dysfunction, partial or complete baldness, or diseases, disorders or conditions that are influenced by intracellular glucocorticoid levels and any combination thereof, adverse effects of glucocorticoid receptor agonist treatment of allergic-inflammatory diseases such as asthma and atopic dermatitis, adverse effects of glucocorticoid receptor agonist treatment of disorders of the respiratory system e.g. asthma, cystic fibrosis, emphysema, bronchitis, hypersensitivity, pneumonitis, eosinophilic pneumonias, pulmonary fibrosis, adverse effects of glucocorticoid receptor agonist treatment of inflammatory bowel disease such as Crohn's disease and ulcerative colitis; adverse effects of glucocorticoid receptor agonist treatment of disorders of the immune system, connective tissue and joints e.g. reactive arthritis, rheumatoid arthritis, Sjögren's syndrome, systemic lupus erythematosus, lupus nephritis, Henoch-Schönlein purpura, Wegener's granulomatosis, temporal arteritis, systemic sclerosis, vasculitis, sarcoidosis, dermatomyositis-polymyositis, pemphigus vulgaris; adverse effects of glucocorticoid receptor agonist treatment of endocrinological diseases such as hyperthyroidism, hypoaldosteronism, hypopituitarism; adverse effects of glucocorticoid receptor agonist treatment of hematological diseases e.g. hemolytic anemia, thrombocytopenia, paroxysmal nocturnal hemoglobinuria; adverse effects of glucocorticoid receptor agonist treatment of cancer such as spinal cord diseases, neoplastic compression of the spinal cord, brain tumours, acute lymphoblastic leukemia, Hodgkin's disease, chemotherapy-induced nausea, adverse effects of glucocorticoid receptor agonist treatment of diseases of muscle and at the neuro-muscular joint e.g. myasthenia gravis and heriditary myopathies (e.g. Duchenne muscular dystrophy), adverse effects of glucocorticoid receptor agonist treatment in the context of surgery & transplantation e.g. trauma, post-surgical stress, surgical stress, renal transplantation, liver transplantation, lung transplantation, pancreatic islet transplantation, blood stem cell transplantation, bone marrow transplantation, heart transplantation, adrenal gland transplantation, tracheal transplantation, intestinal transplantation, corneal transplantation, skin grafting, keratoplasty, lens implantation and other procedures where immunosuppression with glucocorticoid receptor agonists is beneficial; adverse effects of glucocorticoid receptor agonist treatment of brain absess, nausea/vomiting, infections, hypercalcemia, adrenal hyperplasia, autoimmune hepatitis, spinal cord diseases, saccular aneurysms or adverse effects to glucocorticoid receptor agonist treatment in other diseases, disorders and conditions where glucocorticoid receptor agonists provide clinically beneficial effects.

Accordingly, in a further aspect the invention relates to a compound according to the invention for use as a pharmaceutical composition.

The invention also relates to pharmaceutical compositions comprising, as an active ingredient, at least one compound according to the invention together with one or more pharmaceutically acceptable carriers or diluents.

The pharmaceutical composition is preferably in unit dosage form, comprising from about 0.05 mg/day to about 2000 mg/day, preferably from about 1 mg/day to about 500 mg/day of a compound according to the invention.

In another embodiment, the patient is treated with a compound according to the invention for at least about 1 week, for at least about 2 weeks, for at least about 4 weeks, for at least about 2 months or for at least about 4 months.

In yet another embodiment, the pharmaceutical composition is for oral, nasal, transdermal, pulmonal or parenteral administration.

Furthermore, the invention relates to the use of a compound according to the invention for the preparation of a pharmaceutical composition for the treatment, prevention and/or prophylaxis of disorders and diseases wherein a modulation or an inhibition of the activity of 11βHSD1 is beneficial.

The invention also relates to a method for the treatment, prevention and/or prophylaxis of disorders and diseases wherein a modulation or an inhibition of the activity of 11βHSD1 is beneficial, the method comprising administering to a subject in need thereof an effective amount of a compound according to the invention.

In a preferred embodiment of the invention the present compounds are used for the preparation of a medicament for the treatment, prevention and/or prophylaxis of any diseases and conditions that are influenced by intracellular glucocorticoid levels as mentioned above.

Thus, in a preferred embodiment of the invention the present compounds are used for the preparation of a medicament for the treatment, prevention and/or prophylaxis of conditions and disorders where a decreased level of active intracellular glucocorticoid is desirable, such as the conditions and diseases mentioned above.

In yet a preferred embodiment of the invention the present compounds are used for the preparation of a medicament for the treatment, prevention and/or prophylaxis of the metabolic syndrome including insulin resistance, dyslipidemia, hypertension and obesity.

In yet another preferred embodiment of the invention the present compounds are used for the preparation of a medicament for the treatment, prevention and/or prophylaxis of type 2 diabetes, impaired glucose tolerance (IGT), impaired fasting glucose (IFG).

In yet another preferred embodiment of the invention the present compounds are used for the preparation of a pharmaceutical composition for the delaying or prevention of the progression from IGT to type 2 diabetes.

In yet another preferred embodiment of the invention the present compounds are used for the preparation of a pharmaceutical composition for the delaying or prevention of the progression of the metabolic syndrome into type 2 diabetes.

In still another preferred embodiment of the invention the present compounds are used for the preparation of a pharmaceutical composition for the treatment, prevention and/or prophylaxis of diabetic late complications including cardiovascular diseases; arteriosclerosis; atherosclerosis.

In a further preferred embodiment of the invention the present compounds are used for the preparation of a pharmaceutical composition for the treatment, prevention and/or prophylaxis of neurodegenerative and psychiatric disorders.

In yet a further preferred embodiment of the invention the present compounds are used for the preparation of a pharmaceutical composition for the treatment, prevention and/or prophylaxis of adverse effects of glucocorticoid receptor agonist treatment or therapy.

In another embodiment of the present invention, the route of administration may be any route which effectively transports a compound according to the invention to the appropriate or desired site of action, such as oral, nasal, buccal, transdermal, pulmonal, or parenteral.

In still a further aspect of the invention the present compounds are administered in combination with one or more further active substances in any suitable ratios. Such further active substances may e.g. be selected from antiobesity agents, antidiabetics, agents modifying the lipid metabolism, antihypertensive agents, glucocorticoid receptor agonists, agents for the treatment and/or prevention of complications resulting from or associated with diabetes and agents for the treatment and/or prevention of complications and disorders resulting from or associated with obesity.

Thus, in a further aspect of the invention the present compounds may be administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator-activated receptor) modulators, RXR (retinoid X receptor) modulators, TR β agonists, AGRP (Agouti related protein) inhibitors, H3 histamine antagonists, opioid antagonists (such as naltrexone), exendin-4, GLP-1 and ciliary neurotrophic factor.

In one embodiment of the invention the antiobesity agent is leptin; dexamphetamine or amphetamine; fenfluramine or dexfenfluramine; sibutramine; orlistat; mazindol or phentermine.

Suitable antidiabetic agents include insulin, insulin analogues and derivatives such as those disclosed in EP 792 290 (Novo Nordisk A/S), e.g. N^{εB29}-tetradecanoyl des (B30) human insulin, EP 214 826 and EP 705 275 (Novo Nordisk A/S), e.g. Asp^{B28} human insulin, U.S. Pat. No. 5,504,188 (Eli Lilly), e.g. Lys^{B28} Pro^{B29} human insulin, EP 368 187 (Aventis), e.g. Lantus, which are all incorporated herein by reference, GLP-1 (glucagon like peptide-1) and GLP-1 derivatives such as those disclosed in WO 98/08871 to Novo Nordisk A/S, which is incorporated herein by reference as well as orally active hypoglycaemic agents.

The orally active hypoglycaemic agents preferably comprise sulphonylureas, biguanides, meglitinides, glucosidase inhibitors, glucagon antagonists such as those disclosed in WO 99/01423 to Novo Nordisk A/S and Agouron Pharmaceuticals, Inc., GLP-1 agonists, potassium channel openers such as those disclosed in WO 97/26265 and WO 99/03861 to Novo Nordisk A/S which are incorporated herein by reference, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents and antilipidemic agents as PPARα modulators, PPARδ modulators, cholesterol absorption inhibitors, HSL (hormone-sensitive lipase) inhibitors and HMG CoA inhibitors (statins), nicotinic acid, fibrates, anion exchangers, compounds lowering food intake, bile acid resins, RXR agonists and agents acting on the ATP-dependent potassium channel of the β-cells.

In one embodiment, the present compounds are administered in combination with insulin or an insulin analogue or derivative, such as N^{εB29}-tetradecanoyl des (B30) human insulin, Asp^{B28} human insulin, Lys^{B28} Pro^{B29} human insulin, Lantus®, or a mixpreparation comprising one or more of these.

In a further embodiment the present compounds are administered in combination with a sulphonylurea e.g. tolbutamide, glibenclamide, glipizide or glicazide.

In another embodiment the present compounds are administered in combination with a biguanide e.g. metformin.

In yet another embodiment the present compounds are administered in combination with a meglitinide e.g. repaglinide or senaglinide.

In still another embodiment the present compounds are administered in combination with a thiazolidinedione e.g. troglitazone, ciglitazone, pioglitazone, rosiglitazone or compounds disclosed in WO 97/41097 such as 5-[[4-[3-Methyl-4-oxo-3,4-dihydro-2-quinazolinyl]methoxy]phenyl-methyl]thiazolidine-2,4-dione or a pharmaceutically acceptable salt thereof, preferably the potassium salt.

In yet another embodiment the present compounds may be administered in combination with the insulin sensitizers disclosed in WO 99/19313 such as (−)3-[4-[2-Phenoxazin-10-yl)ethoxy]phenyl]-2-ethoxypropanoic acid or a pharmaceutically acceptable salts thereof, preferably the arginine salt.

In a further embodiment the present compounds are administered in combination with an α-glucosidase inhibitor e.g. miglitol or acarbose.

In another embodiment the present compounds are administered in combination with an agent acting on the ATP-dependent potassium channel of the β-cells e.g. tolbutamide, glibenclamide, glipizide, glicazide or repaglinide.

Furthermore, the present compounds may be administered in combination with nateglinide.

In still another embodiment the present compounds are administered in combination with an antihyperlipidemic agent or antilipidemic agent e.g. cholestyramine, colestipol, clofibrate, gemfibrozil, fenofibrate, bezafibrate, tesaglitazar, EML-4156, LY-818, MK-767, atorvastatin, fluvastatin, lovastatin, pravastatin, simvastatin, acipimox, probucol, ezetimibe or dextrothyroxine.

In a further embodiment the present compounds are administered in combination with more than one of the above-mentioned compounds e.g. in combination with a sulphonylurea and metformin, a sulphonylurea and acarbose, repaglinide and metformin, insulin and a sulphonylurea, insulin and metformin, insulin, insulin and lovastatin, etc.

Further, the present compounds may be administered in combination with one or more antihypertensive agents. Examples of antihypertensive agents are β-blockers such as alprenolol, atenolol, timolol, pindolol, propranolol, metoprolol, bisoprololfumerate, esmolol, acebutelol, metoprolol, acebutolol, betaxolol, celiprolol, nebivolol, tertatolol, oxprenolol, amusolalul, carvedilol, labetalol, 132-receptor blockers e.g. Satenolol, OPC-1085, ACE (angiotensin converting enzyme) inhibitors such as quinapril, lisinopril, enalapril, captopril, benazepril, perindopril, trandolapril, fosinopril, ramipril, cilazapril, delapril, imidapril, moexipril, spirapril, temocapril, zofenopril, S-5590, fasidotril, Hoechst-Marion Roussel: 100240 (EP 00481522), omapatrilat, gemopatrilat and GW-660511, calcium channel blockers such as nifedipine, felodipine, nicardipine, isradipine, nimodipine, diltiazem, amlodipine, nitrendipine, verapamil, lacidipine, lercanidipine, aranidipine, cilnidipine, clevidipine, azelnidipine, barnidipine, efonodipine, iasidipine, iemildipine, iercanidipine, manidipine, nilvadipine, pranidipine, furnidipine, α-blockers such as doxazosin, urapidil, prazosin, terazosin, bunazosin and OPC-28326, diuretics such as thiazides/sulphonamides (e.g. bendroflumetazide, chlorothalidone, hydrochlorothiazide and clopamide), loop-diuretics (e.g. bumetanide, furosemide and torasemide) and potassium sparing diuretics (e.g. amiloride, spironolactone), endothelin ET-A antagonists such as ABT-546, ambrisetan, atrasentan, SB-234551, CI-1034, S-0139 and YM-598, endothelin antagonists e.g. bosentan and J-104133, renin inhibitors such as aliskiren, vasopressin V1 antagonists e.g. OPC-21268, vasopressin V2 antagonists such as tolvaptan, SR-121463 and OPC-31260, B-type natriuretic peptide agonists e.g. Nesiritide, angiotensin II antagonists such as irbesartan, candesartancilexetil, losartan, valsartan, telmisartan, eprosartan, candesartan, CL-329167, eprosartan, iosartan, olmesartan, pratosartan, TA-606, and YM-358, 5-HT2 agonists e.g. fenoldopam and ketanserin, adenosine A1 antagonists such as naftopidil, N-0861 and FK-352, thromboxane A2 antagonists such as KT2-962, endopeptidase inhibitors e.g. ecadotril, nitric oxide agonists such as LP-805, dopamine D1 antagonists e.g. MYD-37, dopamine D2 agonists such as nolomirole, n-3 fatty acids e.g. omacor, prostacyclin agonists such as treprostinil, beraprost, PGE1 agonists e.g. ecraprost, Na+/K+ ATPase modulators e.g. PST-2238, Potassium channel activators e.g. KR-30450, vaccines such as PMD-3117, Indapamides, CGRP-unigene, guanylate cyclase stimulators, hydralazines, methyldopa, docarpamine, moxonidine, CoAprovel, MondoBiotech-811.

Further reference can be made to Remington: The Science and Practice of Pharmacy, 19^{th} Edition, Gennaro, Ed., Mack Publishing Co., Easton, Pa., 1995.

Furthermore, the present compounds may be administered in combination with one or more glucocorticoid receptor agonists. Examples of such glucocorticoid receptor agonists are betametasone, dexamethasone, hydrocortisone, methylprednisolone, prednisolone, prednisone, beclomethasone, butixicort, clobetasol, flunisolide, flucatisone (and analogues), momethasone, triamcinolonacetonide, triamcinolonhexacetonide GW-685698, NXC-1015, NXC-1020, NXC-1021, NS-126, P-4112, P-4114, RU-24858 and T-25 series.

It should be understood that any suitable combination of the compounds according to the invention with one or more of the above-mentioned compounds and optionally one or more further pharmacologically active substances are considered to be within the scope of the present invention.

### Pharmaceutical Compositions

The compounds of the present invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as hard or soft capsules, tablets, troches, dragees, pills, lozenges, powders and granules. Where appropriate, they can be prepared with coatings such as enteric coatings or they can be formulated so as to provide controlled release of the active ingredient such as sustained or prolonged release according to methods well-known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, crmes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 2000 mg, e.g. from about 0.1 to about 1000 mg, from about 0.5 mg to about 500 mg., from about 1 mg to about 200 mg, e.g. about 100 mg.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. Examples are an acid addition salt of a compound having the utility of a free base and a base addition salt of a compound having the utility of a free acid. The term “pharmaceutically acceptable salts” refers to non-toxic salts of the compounds for use according to the present invention which are generally prepared by reacting the free base with a suitable organic or inorganic acid or by reacting the acid with a suitable organic or inorganic base. When a compound for use according to the present invention, contains a free base such salts are prepared in a conventional manner by treating a solution or suspension of the compound with a chemical equivalent of a pharmaceutically acceptable acid. When a compounds for use according to the present invention, contains a free acid such salts are prepared in a conventional manner by treating a solution or suspension of the compound with a chemical equivalent of a pharmaceutically acceptable base. Physiologically acceptable salts of a compound with a hydroxy group include the anion of said compound in combination with a suitable cation such as sodium or ammonium ion. Other salts which are not pharmaceutically acceptable may be useful in the preparation of compounds for use according to the present invention and these form a further aspect of the present invention.

For parenteral administration, solutions of the present compounds in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of suitable carriers are water, salt solutions, alcohols, polyethylene glycols, polyhydroxyethoxylated castor oil, peanut oil, olive oil, syrup, phospholipids, gelatine, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents.

The pharmaceutical compositions formed by combining the compounds of the invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

Compositions intended for oral use may be prepared according to any known method, and such compositions may contain one or more agents selected from the group consisting of sweetening agents, flavouring agents, colouring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, incorporated herein by reference, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsule wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions comprising a compound for use according to the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, preservative and flavouring and colouring agent. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the present invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the present invention are contemplated. For the purpose of this application, topical applications shall include mouth washes and gargles.

The compounds for use according to the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In addition, some of the compounds for use according to the present invention may form solvates with water or common organic solvents. Such solvates are also encompassed within the scope of the present invention.

Thus, in a further embodiment, there is provided a pharmaceutical composition comprising a compound for use according to the present invention, or a pharmaceutically acceptable salt, solvate, or prodrug thereof, and one or more pharmaceutically acceptable carriers, excipients, or diluents.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet which may be prepared by conventional tabletting techniques may contain:

Core:

| | | |
|---|---|---|
| Active compound (as free compound or | 5.0 | mg |
| salt thereof) | | |
| Lactosum Ph. Eur. | 67.8 | mg |
| Cellulose, microcryst. (Avicel) | 31.4 | mg |
| Amberlite ®IRP88* | 1.0 | mg |

| | |
|---|---|
| Magnesii stearas Ph. Eur. | q.s. |

Coating:

| | | |
|---|---|---|
| Hydroxypropyl methylcellulose | approx. 9 | mg |
| Mywacett 9-40 T** | approx. 0.9 | mg |

*Polacrillin potassium NF, tablet disintegrant, Rohm and Haas.
**Acylated monoglyceride used as plasticizer for film coating.

The compounds of the invention may be administered to a patient which is a mammal, especially a human in need thereof. Such mammals include also animals, both domestic animals, e.g. household pets, and non-domestic animals such as wildlife.

Any novel feature or combination of features described herein is considered essential to this invention.

The present invention also relate to the below methods of preparing the compounds of the invention.

The features disclosed in the foregoing description may, both separately and in any combination thereof, be material for realising the invention in diverse forms thereof.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way,

Any combination of the above-described elements in all possible variations thereof is encompassed by the invention unless otherwise indicated herein or otherwise clearly con-tradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also pro-vide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents,

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a formulation described herein as comprising a particular element should be understood as also describing a formulation consisting of that element, unless otherwise stated or clearly contradicted by context).

This invention includes all modifications and equivalents of the subject matter recited in the aspects or claims presented herein to the maximum extent permitted by applicable law.

The present invention is further illustrated in the following representative examples which are, however, not intended to limit the scope of the invention in any way.

### EXAMPLES

The following general procedures refer to intermediate compounds and final products for general formula (I) identified in the specification and in the synthesis schemes. The preparation of the compounds of general formula (I) of the present invention is described in detail using the following. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, which is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials. The structures of the compounds are confirmed by either elemental analysis or nuclear magnetic resonance (NMR), where peaks assigned to characteristic protons in the title compounds are presented where appropriate. ¹H NMR shifts (δH) are given in parts per million (ppm) down field from tetramethylsilane as internal reference standard. M.p.: is melting point and is given in ° C. and is not corrected. Column chromatography was carried out using the technique described by W. C. Still et al., J. Org. Chem. 43: 2923 (1978) on Merck silica gel 60 (Art. 9385).

### HPLC Systems:

HPLC-MS: The RP-analysis was performed on an Agilent HPLC system (1100 degasser, 1100 pump, 1100 injector and a 1100 DAD) fitted with an Agilent MS detector system Model VL (MW 0-1000) and a S.E.D.E.R.E Model Sedex 55 ELS detector system using a Waters X-terra MS C18 column (5 μm, 3.0 mm×50 mm) with gradient elution, 5% to 95% solvent B (0.05% TFA in acetonitrile) in solvent A (0.05% TFA in water) within 3 min, 2.7 mL/min.

The abbreviations as used in the present application have the following meaning:
TLC: Thin layer chromatography
CDCl₃: Deuterio chloroform

### DCM: Dichloromethane

### DIIC: N,N′-Diisopropylcarbodiimide

### DMAP: 4-Dimethylaminopyridine

DMSO-d₆: Hexadeuterio dimethylsulfoxide

### DMSO: Dimethylsulfoxide

### DIPEA: Diisopropylethylamine

EDAC: 1-(3-Dimethylaminopropyl)-3-ethylcarbodiimide hydrochloride
EtOAc: Ethyl acetate

### THF: Tetrahydrofuran

### DMF: N,N-dimethylformamide

### HOBT: 1-Hydroxy-benzotriazole

### POL: Polystyrene

Ptg: Protecting group

### MeCN: Acetonitrile

### NMP: N-Methylpyrrolidinone

### TEA: Triethylamine

TFA: Trifluoroacetic acid
EDAC: 1-(3-Dimethylaminopropyl)-3-ethylcarbodiimide, hydrochloride
min: minutes
hrs: hours

9H-Carbazole-3-carboxylic acid and the corresponding N-methyl and N-ethyl analogues are commercially available.

Analogues with R⁴ different from hydrogen are prepared in a similar way as described in J. Med. Chem. 2002, 45, 3509-3523.

Dibenzothiophene-2-carboxylic acid can be prepared as described in J. Org. Chem. 1938, 3, 108.

9H-Fluorene-3-carboxylic acid is commercially available and 9-oxo-9H-fluorene-3-carboxylic acid is obtained via oxidation of the former.

Dibenzofuran-2-carboxylic acid is obtained as described in J. Ami. Chem. Soc. 1939, 61, 2836-2842.

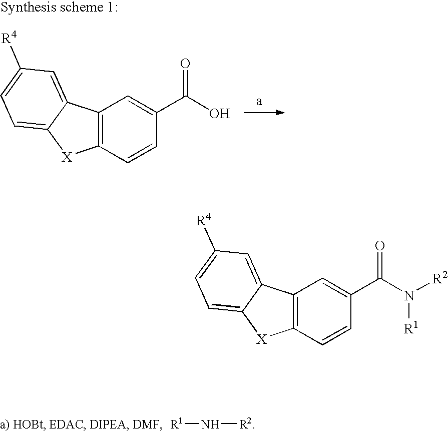

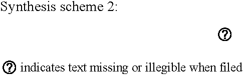

### Example 1

### 9-Methyl-9H-carbazole-3-carboxylic acid (5-hydroxy-adamantan-2-yl)-methyl-amide

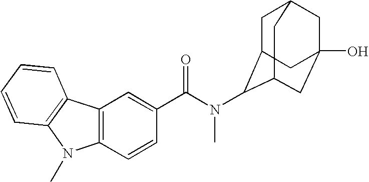

### 4-Methylamino-adamantan-1-ol

### Step-A: (5-Hydroxyadamantan-2-yl)carbamic acid tert-butyl ester

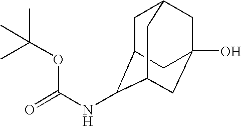

Ammonium formate (10 g, 0.15 mol) was added to a solution of 5-hydroxy-adamantan-2-one (4.5 g, 0.027 mol) in MeOH (50 ml). Then 10% Pd—C (500 mg) was added carefully and the solution heated under reflux for 1 h. It was then filtered through celite and to the filtrate at 0° C. was added triethylamine (11.2 mL, 0.081 mol) and Boc anhydride (7.06 g, 0.0324 mol). The solution was stirred for 4 hrs at RT and then concentrated under reduced pressure. The residue was diluted with water and extracted with EtOAc. The organic layer was dried (MgSO₄) and concentrated in vacuo which afforded 7 g (96%) of (5-hydroxy-adamantan-2-yl)carbamic acid tert-butyl ester).

LC/MS: 168 (M+1)

¹H-NMR (300 MHz, DMSO-d₆): δ 6.8 (d, 1H), 6.7 (brs, 1H), 3.45 (d, 1H), 2.0 (s, 1H), 1.75-1.95 (m, 4H), 1.5-1.7 (m, 6H), 1.35 (s, 9H), 1.25 (t, 2H).

### Step B: 4-Methylamino-adamantan-1-ol

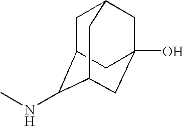

Lithium aluminium hydride (0.711 g, 0.018 mol) was added to a solution of (5-hydroxy-adamantan-2-yl)carbamic acid tert-butyl ester (1 g, 3.7 mmol) in THF (50 mL) at 0° C. under a nitrogen atmosphere. The slurry was heated under reflux for 5 hrs. It was then cooled to 0° C. and quenched with 30% NaOH soln (12 mL) and filtered. The filtrate was concentrated in vacuo to give 0.6 g (90%) of 4-methylamino-adamantan-1-ol as a white solid.

LC/MS: 181.9 (M+1)

¹H-NMR (300 MHz, DMSO-d₆): δ 4.3 (s, 1H), 4.2 (s, 1H), 2.4 (s, 0.7H), 2.3 (s, 0.3H), 2.2 (s, 3H), 1.8-2.0 (m, 5H), 1.5-1.6 (m, 5H), 1.4-1.5 (m, 2H), 1.2 (m, 2H).

To a solution of 9H-carbazole-3-carboxylic acid (220 mg, 1.04 mmol) in DMF (3 mL) was added HOBt (155 mg, 1.15 mmol) and EDAC (220 mg, 1.15 mmol). The mixture was stirred at room temperature for 15 min at which time 4-methylamino-adamantan-1-ol (208 mg, 1.15 mmol) and DIPEA (545 μL, 3.13 mmol) were added. After stirring for 16 hrs at room temperature the volatiles were evaporated in vacuo and the residue dissolved in AcOEt (10 mL). The organic phase was washed with aqueous NaHCO₃, dried (MgSO4), filtered and evaporated in vacuo. The residue was purified on prep. HPLC affording 160 mg (41%) of the title compound as a solid.

¹H-NMR (400 MHz, DMSO-d₆) δ 1.42-1.58 (m, 2H), 1.59-1.77 (m, 5H), 1.91-2.20 (m, 2H), 2.43 (br.s., 1H), 3.10 (d, 2H), 3.35 (s, 3H), 4.00+4.07 (2×br.s., 1H), 4.45+4.51 (2×s, 1H), 7.19 (t, 1H), 7.36-7.57 (m, 4H), 8.16-8.29 (m, 2H), 11.46 (br.s., 1H).

The compounds are prepared according to the synthesis scheme 1 or 2 by the use of standard reactions readily recognized by those skilled in the art.

| | | | LC-MS |
|---|---|---|---|
| Ex | | | (electro- |
| No. | Molecule | Name (IUPAC) | spray) |
| 1 | 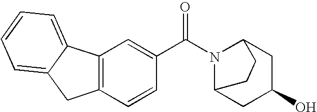 | (9H-Fluoren-3-yl)-(cis 3- hydroxy-8-aza-bicyclo[3.2.1]oct- 8-yl)-methanone | 320 |
| 2 | 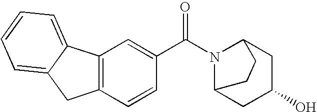 | (9H-Fluoren-3-yl)-(trans 3- hydroxy-8-aza-bicyclo[3.2.1]oct- 8-yl)-methanone | 320 |
| 3 | 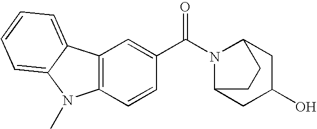 | (3-Hydroxy-8-aza-bicyclo[3.2.1]- oct-8-yl)-(9-methyl-9H-carbazol- 3-yl)-methanone | 335 |
| 4 | 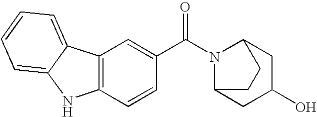 | (9H-Carbazol-3-yl)-(3-hydroxy- 8-aza-bicyclo[3.2.1]oct-8-yl)- methanone | 321 |
| 5 | 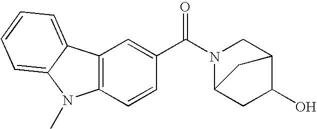 | (5-Hydroxy-2-aza-bicyclo[2.2.1]- hept-2-yl)-(9-methyl-9H- carbazol-3-yl)-methanone | 322 |
| 6 | 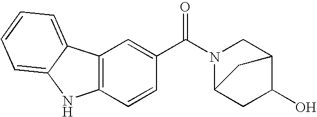 | (9H-Carbazol-3-yl)-(5-hydroxy- 2-aza-bicyclo[2.2.1]hept-2-yl)- methanone | 306 |
| 7 | 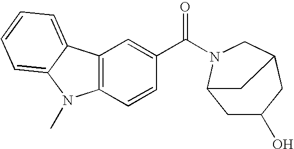 | (3-Hydroxy-6-aza-bicyclo[3.2.1]- oct-6-yl)-(9-methyl-9H-carbazol- 3-yl)-methanone | 334 |
| 8 | 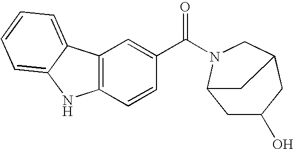 | (9H-Carbazol-3-yl)-(3-hydroxy- 6-aza-bicyclo[3.2.1]oct-6-yl)- methanone | 321 |
| 9 | 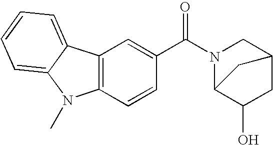 | (6-Hydroxy-2-aza-bicyclo[2.2.1]- hept-2-yl)-(9-methyl-9H- carbazol-3-yl)-methanone | 321 |
| 10 | 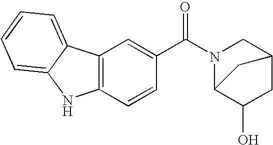 | (9H-Carbazol-3-yl)-(6-hydroxy- 2-aza-bicyclo[2.2.1]hept-2-yl)- methanone | 307 |
| 11 | 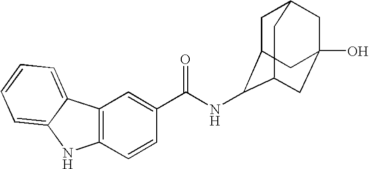 | 9H-Carbazole-3-carboxylic acid (5-hydroxy-adamantan-2-yl)- amide | 361 |
| 12 | 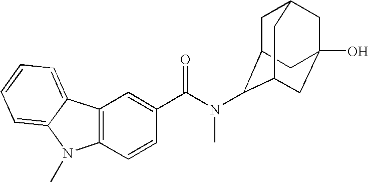 | 9-Methyl-9H-carbazole-3- carboxylic acid (5-hydroxy- adamantan-2-yl)-methyl-amide | 389 |
| 13 | 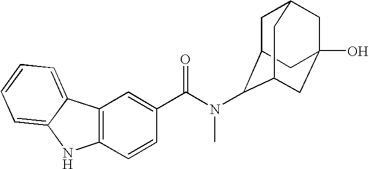 | 9H-Carbazole-3-carboxylic acid (5-hydroxy-adamantan-2-yl)- methyl-amide | 375 |
| 14 | 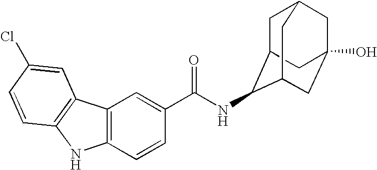 | 6-Chloro-9H-carbazole-3- carboxylic acid (trans 5-hydroxy- adamantan-2-yl)-amide | 395 |
| 15 | 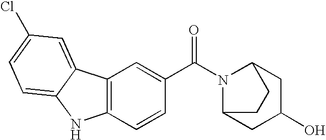 | (6-Chloro-9H-carbazol-3-yl)-(3- hydroxy-8-aza-bicyclo[3.2.1]oct- 8-yl)-methanone | 355 |
| 16 | 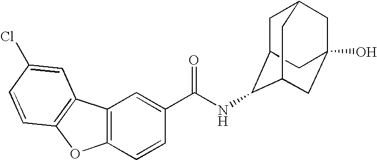 | 6-Chloro-9H-carbazole-3- carboxylic acid (cis 5-hydroxy- adamantan-2-yl)-amide | 395 |
| 17 | 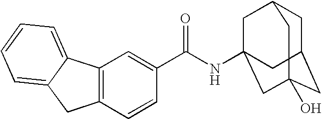 | 9H-Fluorene-3-carboxylic acid (3-hydroxy-adamantan-1-yl)- amide | 360 |
| 18 | 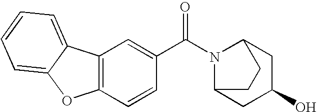 | Dibenzofuran-2-yl-(3-hydroxy-8- aza-bicyclo[3.2.1]oct-8-yl)- methanone | 322 |
| 19 | 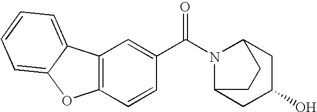 | Dibenzofuran-2-yl-(3-hydroxy-8- aza-bicyclo[3.2.1]oct-8-yl)- methanone | 322 |
| 20 | 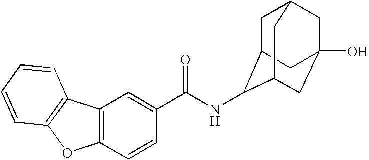 | Dibenzofuran-2-carboxylic acid (5-hydroxy-adamantan-2-yl)- amide | 362 |
| 21 | 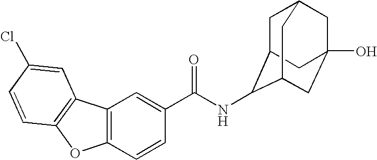 | 8-Chloro-dibenzofuran-2- carboxylic acid (5-hydroxy- adamantan-2-yl)-amide | 396 |
| 22 | 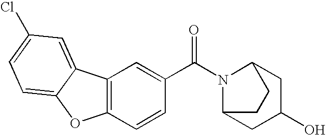 | (8-Chloro-dibenzofuran-2-yl)-(3- hydroxy-8-aza-bicyclo[3.2.1]oct- 8-yl)-methanone | 245 |
| 23 | 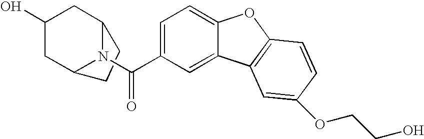 | (3-Hydroxy-8-aza- bicyclo[3.2.1]oct-8-yl)-[8-(2- hydroxy-ethoxy)-dibenzofuran-2- yl]-methanone | 382 |
| 24 | 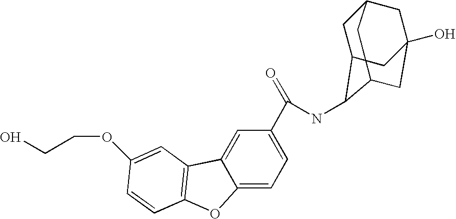 | 8-(2-Hydroxy-ethoxy)- dibenzofuran-2-carboxylic acid (5-hydroxy-adamantan-2-yl)- amide | 422 |

### Pharmacological Methods

### 11βHSD1 Enzyme Assay

### Materials

³H-cortisone and anti-rabbit Ig coated scintillation proximity assay (SPA) beads were purchased from Amersham Pharmacia Biotech, β-NADPH was from Sigma and rabbit anti-cortisol antibodies were from Fitzgerald. An extract of yeast transformed with h-11βHSD1 (Hutt et al., FEBS Lett, 441, 25 (1998)) was used as the source of enzyme. The test compounds were dissolved in DMSO (10 mM). All dilutions were performed in a buffer containing 50 mM TRIS-HCl (Sigma Chemical Co), 4 mM EDTA (Sigma Chemical Co), 0.1% BSA (Sigma Chemical Co), 0.01% Tween-20 (Sigma Chemical Co) and 0.005% bacitracin (Novo Nordisk A/S), pH=7.4. Optiplate 96 wells plates were supplied by Packard. The amount of ³H-cortisol bound to the SPA beads was measured on TopCount NXT, Packard.

### Methods

h-11βHSD1, 120 nM ³H-cortisone, 4 mM β-NADPH, antibody (1:200), serial dilutions of test compound and SPA particles (2 mg/well) were added to the wells. The reaction was initiated by mixing the different components and was allowed to proceed under shaking for 60 min at 30° C. The reaction was stopped be the addition of 10 fold excess of a stopping buffer containing 500 μM carbenoxolone and 1 μM cortisone. Data was analysed using GraphPad Prism software.

TABLE 1
Inhibition of h-11βHSD1 by compounds of the invention

| | h-11βHSD1 |
|---|---|
| | IC₅₀ values |
| Example No. | (nM) |
| 1 | 8 |
| 2 | 246 |
| 3 | 6 |
| 4 | 30 |
| 5 | 64 |
| 7 | 360 |
| 9 | 274 |
| 10 | 1029 |
| 11 | 29 |
| 12 | 41 |
| 13 | 76 |

While the invention has been described and illustrated with reference to certain preferred embodiments thereof, those skilled in the art will appreciate that various changes, modifications, and substitutions can be made therein without departing from the spirit and scope of the present invention. For example, effective dosages other than the preferred dosages as set forth herein may be applicable as a consequence of variations in the responsiveness of the mammal being treated for the disease(s). Likewise, the specific pharmacological responses observed may vary according to and depending on the particular active compound selected or whether there are present pharmaceutical carriers, as well as the type of formulation and mode of administration employed, and such expected variations or differences in the results are contemplated in accordance with the objects and practices of the present invention. Accordingly, the invention is not to be limited as by the appended claims.

The features disclosed in the foregoing description and/or in the claims may both separately and in any combination thereof be material for realising the invention in diverse forms thereof.

Features of the Invention:

1. A compound of the general formula (I):

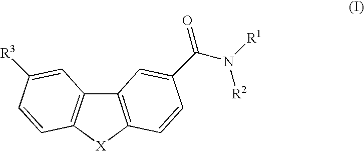

wherein

### X is CR⁴R⁵, C═O, NR⁴, O, S, or SO₂;

R¹ is hydrogen or C₁-C₄alkyl;
R² is a monovalent radical having one of the following formulae, wherein the symbol (*) denotes the point of attachment:

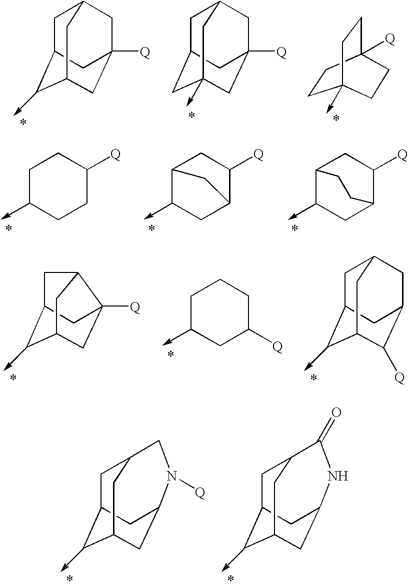

wherein Q is hydroxy, hydroxymethyl, carboxy, —C(═O)—NR⁴R⁵, —S(═O)₂NR⁴R⁶, or S(═O)₂R⁶; or
R¹ and R² together with the nitrogen to which they are attached forms one of the following formulae wherein the symbol (*) denotes the point of attachment:

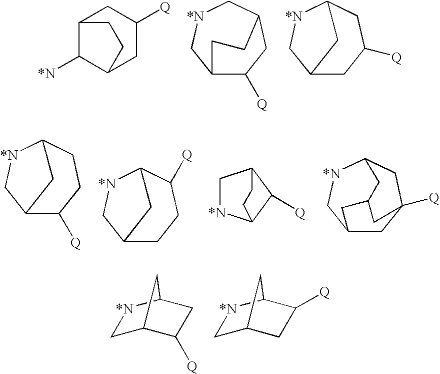

wherein Q is hydroxy, hydroxymethyl, carboxy, —C(═O)—NR⁴R⁵, —S(═O)₂NR⁴R⁵, or S(═O)₂R⁶;
R³ is hydrogen, halogen, hydroxy, cyano, C₁-C₄alkyl, aryl, heteroaryl, —NR⁴R⁵, —OR⁶, —SR⁶, or SO₂R⁶, wherein said alkyl, aryl and heteroaryl groups are optionally substituted with one or two independently selected R⁷;
R⁴ and R⁵ independently are hydrogen or C₁-C₄alkyl, wherein said C₁-C₄alkyl is optionally substituted with one or two independently selected R⁷;
R⁶ is hydrogen, C₁-C₄alkyl; or
R⁷ is selected from the group consisting of hydrogen, cyano, C₁-C₄alkyl, cyclopropyl, hydroxy, halogen, trifluoromethyl, —CH₂OH and carboxy; or
a salt thereof with a pharmaceutically acceptable acid or base, or any optical isomer or mixture of optical isomers, including a racemic mixture, or any tautomeric forms.
2. The compound according to clause 1, wherein X is CR⁴R⁵, C═O or NR⁴.
3. The compound according to clause 2, wherein X is CR⁴R⁵.
4. The compound according to clause 3, wherein X is CH₂.
5. The compound according to clause 2, wherein X is NR⁴.
6. The compound according to clause 5, wherein X is NH or NCH₃.
7. The compound according to any of the preceding clauses, wherein R¹ is hydrogen.
8. The compound according to any of the clauses 1-6, wherein R¹ is C₁-C₄alkyl optionally substituted with one or two independently selected halogen.
9. The compound according to clause 8, wherein R¹ is methyl.
10. The compound according to any of the preceding clauses, wherein Q is hydroxy, hydroxymethyl or carboxy.
11. The compound according to clause 10, wherein Q is hydroxy.
12. The compound according to any of the preceding clauses, wherein R² is a monovalent radical having one of the following formulae, wherein the symbol (*) denotes the point of attachment:

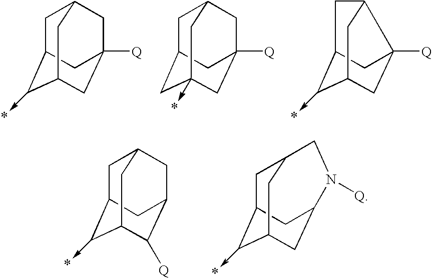

13. The compound according to clause 12, wherein R² is a monovalent radical having one of the following formulae, wherein the symbol (*) denotes the point of attachment:

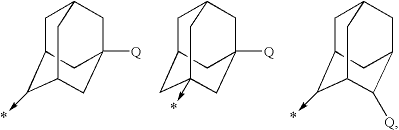

wherein Q is hydroxy.
14. The compound according to any of the clauses 1-6, 10-11, wherein R¹ and R² together with the nitrogen to which they are attached forms one of the following formulae wherein the symbol (*) denotes the point of attachment:

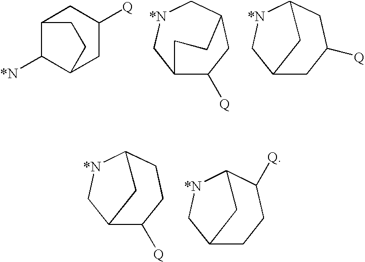

15. The compound according to clause 14, wherein R¹ and R² together with the nitrogen to which they are attached forms one of the following formulae wherein the symbol (*) denotes the point of attachment:

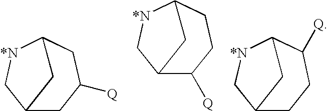

16. The compound according to clause 14, wherein R¹ and R² together with the nitrogen to which they are attached forms the following formula wherein the symbol (*) denotes

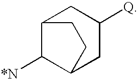

17. The compound according to clause 16, wherein R¹ and R² together with the nitrogen to which they are attached forms the following formula wherein the symbol (*) denotes

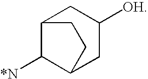

18. The compound according to any of the preceding clauses, wherein R³ is hydrogen, halogen, hydroxy, cyano, or C₁-C₄alkyl, wherein the alkyl is optionally substituted with one or two independently selected R⁷.
19. The compound according to clause 18 wherein R³ is hydrogen.
20. The compound according to any of the preceding clauses, wherein R⁴ is hydrogen.
21. The compound according to any of the clauses 1-19, wherein R⁴ is C₁-C₄alkyl.
22. The compound according to any of the preceding clauses wherein R⁵ is hydrogen.
23. The compound according to any of the preceding clauses wherein R⁶ is hydrogen.
24. The compound according to any of the preceding clauses, wherein R⁷ is hydrogen, hydroxy or halogen.
25. The compound according to any of the preceding clauses, which is selected from the group consisting of
(9H-Fluoren-3-yl)-(trans 3-hydroxy-8-aza-bicyclo[3.2.1]oct-8-yl)-methanone;
(9H-Fluoren-3-yl)-(cis 3-hydroxy-8-aza-bicyclo[3.2.1]oct-8-yl)-methanone;
(3-Hydroxy-8-aza-bicyclo[3.2.1]oct-8-yl)-(9-methyl-9H-carbazol-3-yl)-methanone;
(9H-Carbazol-3-yl)-(3-hydroxy-8-aza-bicyclo[3.2.1]oct-8-yl)-methanone;
(5-Hydroxy-2-aza-bicyclo[2.2.1]hept-2-yl)-(9-methyl-9H-carbazol-3-yl)-methanone;

(9H-Carbazol-3-yl)-(5-hydroxy-2-aza-bicyclo[2.2.1]hept-2-yl)-methanone;
(3-Hydroxy-6-aza-bicyclo[3.2.1]oct-6-yl)-(9-methyl-9H-carbazol-3-yl)-methanone
(9H-Carbazol-3-yl)-(3-hydroxy-6-aza-bicyclo[3.2.1]oct-6-yl)-methanone;
(6-Hydroxy-2-aza-bicyclo[2.2.1]hept-2-yl)-(9-methyl-9H-carbazol-3-yl)-methanone
(9H-Carbazol-3-yl)-(6-hydroxy-2-aza-bicyclo[2.2.1]hept-2-yl)-methanone;
9H-Carbazole-3-carboxylic acid (5-hydroxy-adamantan-2-yl)-amide;
9-Methyl-9H-carbazole-3-carboxylic acid (5-hydroxy-adamantan-2-yl)-methyl-amide;
9H-Carbazole-3-carboxylic acid (5-hydroxy-adamantan-2-yl)-methyl-amide
6-Chloro-9H-carbazole-3-carboxylic acid (trans 5-hydroxy-adamantan-2-yl)-amide;
(6-Chloro-9H-carbazol-3-yl)-(3-hydroxy-8-aza-bicyclo[3.2.1]oct-8-yl)-methanone;
6-Chloro-9H-carbazole-3-carboxylic acid (cis 5-hydroxy-adamantan-2-yl)-amide;
9H-Fluorene-3-carboxylic acid (3-hydroxy-adamantan-1-yl)-amide;
Dibenzofuran-2-yl-(3-hydroxy-8-aza-bicyclo[3.2.1]oct-8-yl)-methanone;
Dibenzofuran-2-yl-(3-hydroxy-8-aza-bicyclo[3.2.1]oct-8-yl)-methanone;
Dibenzofuran-2-carboxylic acid (5-hydroxy-adamantan-2-yl)-amide;
8-Chloro-dibenzofuran-2-carboxylic acid (5-hydroxy-adamantan-2-yl)-amide;
(8-Chloro-dibenzofuran-2-yl)-(3-hydroxy-8-aza-bicyclo[3.2.1]oct-8-yl)-methanone;
   or a prodrug thereof, a salt thereof with a pharmaceutically acceptable acid or base, or any optical isomer or mixture of optical isomers, including a racemic mixture, or any tautomeric forms.
   26. The compound according to any one of the preceding clauses, wherein polar surface area (PSA) of said compound is in the range from 40 Å² to 130 Å², preferably from 50 Å² to 130 Å², more preferably from 60 Å² to 120 Å², more preferably from 70 Å² to 120 Å², most preferable from 70 Å² to 110 Å².
   27. The compound according to any one of the preceding clauses, wherein the molar weight of said compound is in the range from 350D to 650D, preferably from 400D to 600D.
   28. The compound according to any one of the preceding clauses, which is an agent useful for the treatment, prevention and/or prophylaxis of any conditions, disorders and diseases wherein a modulation or an inhibition of the activity of 11βHSD1 is beneficial.
   29. The compound according to any one of the clauses 1-27, which is an agent useful for the treatment, prevention and/or prophylaxis of any conditions, disorders and diseases that are influenced by intracellular glucocorticoid levels.
   30. The compound according to any one of the clauses 1-27 which is an agent useful for the treatment, prevention and/or prophylaxis of conditions, disorders or diseases selected from the group consisting of the metabolic syndrome, insulin resistance, dyslipidemia, hypertension and obesity.
   31. The compound according to any one of the clauses 1-27 which is an agent useful for the treatment, prevention and/or prophylaxis of type 2 diabetes, impaired glucose tolerance (IGT), impaired fasting glucose (IFG).
   32. The compound according to any one of the clauses 1-27 which is an agent useful for the delaying or prevention of the progression from IGT into type 2 diabetes.
   33. The compound according to any one of the clauses 1-27 which is an agent useful for delaying or prevention of the progression of the metabolic syndrome into type 2 diabetes.
   34. The compound according to any one of the clauses 1-27 which is an agent useful for the treatment, prevention and/or prophylaxis of adverse effects of glucocorticoid receptor agonist treatment or therapy.
   35. A pharmaceutical composition comprising, as an active ingredient, at least one compound according to any one of the clauses 1-27 together with one or more pharmaceutically acceptable carriers or excipients.
   36. The pharmaceutical composition according to clause 35 which is for oral, nasal, buccal, transdermal, pulmonal or parenteral administration.
   37. The pharmaceutical composition according to clause 35 or 36 in unit dosage form, comprising from 0.05 mg to 2000 mg/day, from 0.1 mg to 1000 mg or from 0.5 mg to 500 mg per day of the compound according to anyone of the clauses 1-22.
   38. Use of a compound according to any of the clauses 1-27, for the preparation of a pharmaceutical composition for the treatment, prevention and/or prophylaxis of any conditions, disorders and diseases wherein a modulation or an inhibition of the activity of 11βHSD1 is beneficial.
   39. Use of a compound according to any of the clauses 1-27, for the preparation of a pharmaceutical composition for the treatment, prevention and/or prophylaxis of any conditions, disorders and diseases that are influenced by intracellular glucocorticoid levels.
   40. Use of a compound according to any of the clauses 1-27, for the preparation of a pharmaceutical composition for the treatment, prevention and/or prophylaxis of conditions, disorders or diseases selected from the group consisting of the metabolic syndrome, insulin resistance, dyslipidemia, hypertension and obesity.
   41. Use of a compound according to any of the clauses 1-27, for the preparation of a pharmaceutical composition for the treatment, prevention and/or prophylaxis of type 2 diabetes, impaired glucose tolerance (IGT), impaired fasting glucose (IFG).
   42. Use of a compound according to any of the clauses 1-27, for the preparation of a pharmaceutical composition for the delaying or prevention of the progression from IGT to type 2 diabetes.
   43. Use of a compound according to any of the clauses 1-27, for the preparation of a pharmaceutical composition for the delaying or prevention of the progression of the metabolic syndrome into type 2 diabetes.
   44. Use of a compound according to any of the clauses 1-27, for the preparation of a pharmaceutical composition for the treatment, prevention and/or prophylaxis of adverse effects of glucocorticoid receptor agonist treatment or therapy.
   45. A method for the treatment, prevention and/or prophylaxis of any conditions, disorders or diseases wherein a modulation or an inhibition of the activity of 11βHSD1 is beneficial, the method comprising administering to a subject in need thereof an effective amount of a compound according to the invention.
   46. The method according to clause 45 wherein the conditions, disorders or diseases are selected from the group consisting of the metabolic syndrome, insulin resistance, dyslipidemia, hypertension and obesity.

1. A compound of the general formula (I): 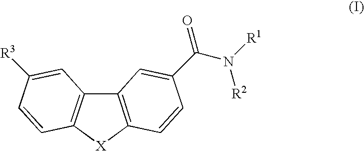
wherein
X is CR⁴R⁵, C═O, NR⁴, O, S, or SO₂;
R¹ is hydrogen or C₁-C₄alkyl;
R² is a monovalent radical having one of the following formulae, wherein the symbol (*) denotes the point of attachment: 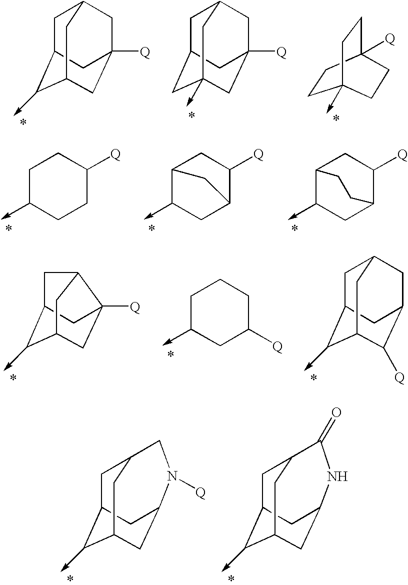
wherein Q is hydroxy, hydroxymethyl, carboxy, —C(═O)—NR⁴R⁵, —S(═O)₂NR⁴R⁵, or S(═O)₂R⁶; or
R¹ and R² together with the nitrogen to which they are attached forms one of the following formulae wherein the symbol (*) denotes the point of attachment: 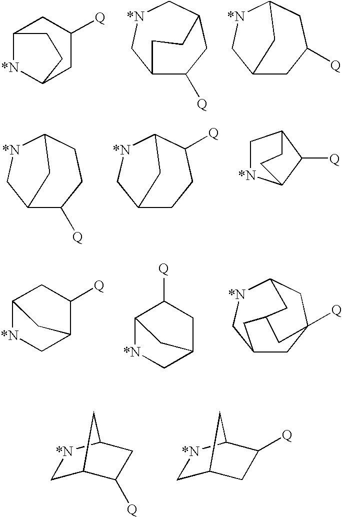
wherein Q is hydroxy, hydroxymethyl, carboxy, —C(═O)—NR⁴R⁵, —S(═O)₂NR⁴R⁵, or S(═O)₂R⁶;
R³ is hydrogen, halogen, hydroxy, cyano, C₁-C₄alkyl, aryl, heteroaryl, —NR⁴R⁵, —OR⁶, —SR⁶, or SO₂R⁶, wherein said alkyl, aryl and heteroaryl groups are optionally substituted with one or two independently selected R⁷;
R⁴ and R⁵ independently are hydrogen or C₁-C₄alkyl, wherein said C₁-C₄alkyl is optionally substituted with one or two independently selected R⁷;
R⁶ is hydrogen, C₁-C₄alkyl, wherein said C₁-C₄alkyl is optionally substituted with hydroxy; or
R⁷ is selected from the group consisting of hydrogen, cyano, C₁-C₄alkyl, cyclopropyl, hydroxy, halogen, trifluoromethyl, CH₂OH and carboxy; or
a salt thereof with a pharmaceutically acceptable acid or base, or any optical isomer or mixture of optical isomers, including a racemic mixture, or any tautomeric forms.

2. A compound of the general formula (I): 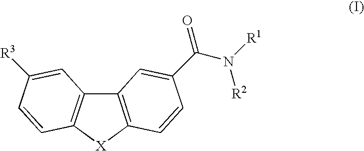
wherein
X is CR⁴R⁵, C═O, NR⁴, O, S, or SO₂;
R¹ is hydrogen or C₁-C₄alkyl;
R² is a monovalent radical having one of the following formulae, wherein the symbol (*) denotes the point of attachment: 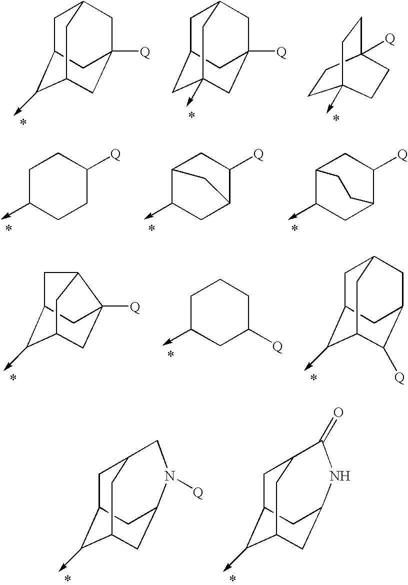
wherein Q is hydroxy, hydroxymethyl, carboxy, —C(═O)—NR⁴R⁵, —S(═O)₂NR⁴R⁵, or S(═O)₂R⁶; or
R¹ and R² together with the nitrogen to which they are attached forms one of the following formulae wherein the symbol (*) denotes the point of attachment: 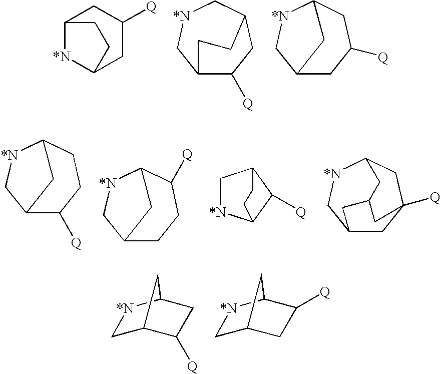
wherein Q is hydroxy, hydroxymethyl, carboxy, —C(═O)—NR⁴R⁵, —S(═O)₂NR⁴R⁵, or S(═O)₂R⁶;
R³ is hydrogen, halogen, hydroxy, cyano, C₁-C₄alkyl, aryl, heteroaryl, —NR⁴R⁵, —OR⁶, —SR⁶, or SO₂R⁶, wherein said alkyl, aryl and heteroaryl groups are optionally substituted with one or two independently selected R⁷;
R⁴ and R⁵ independently are hydrogen or C₁-C₄alkyl, wherein said C₁-C₄alkyl is optionally substituted with one or two independently selected R⁷;
R⁶ is hydrogen, C₁-C₄alkyl; or
R⁷ is selected from the group consisting of hydrogen, cyano, C₁-C₄alkyl, cyclopropyl, hydroxy, halogen, trifluoromethyl, CH₂OH and carboxy; or
a salt thereof with a pharmaceutically acceptable acid or base, or any optical isomer or mixture of optical isomers, including a racemic mixture, or any tautomeric forms.

3. The compound according to claim 1 wherein X is CR⁴R⁵ or NR⁴.

4. The compound according to claim 1 wherein Q is hydroxy.

5. The compound according to claim 1, wherein R² is a monovalent radical having one of the following formulae, wherein the symbol (*) denotes the point of attachment:

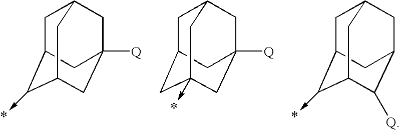

6. The compound according claim 1, wherein R¹ and R² together with the nitrogen to which they are attached forms one of the following formulae wherein the symbol (*) denotes the point of attachment:

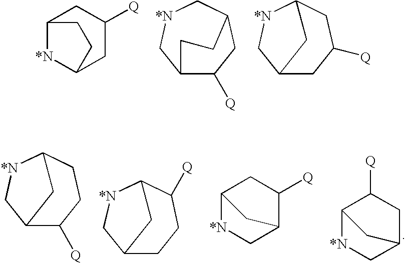

7. The compound according to claim 1, which is selected from the group consisting of
(9H-Fluoren-3-yl)-(trans 3-hydroxy-8-aza-bicyclo[3.2.1]oct-8-yl)-methanone;
(9H-Fluoren-3-yl)-(cis 3-hydroxy-8-aza-bicyclo[3.2.1]oct-8-yl)methanone;
(3-Hydroxy-8-aza-bicyclo[3.2.1]oct-8-yl)-(9-methyl-9H-carbazol-3-yl)-methanone;
(9H-Carbazol-3-yl)-(3-hydroxy-8-aza-bicyclo[3.2.1]oct-8-yl)-methanone;
(5-Hydroxy-2-aza-bicyclo[2.2.1]hept-2-yl)-(9-methyl-9H-carbazol-3-yl)-methanone;
(9H-Carbazol-3-yl)-(5-hydroxy-2-aza-bicyclo[2.2.1]hept-2-yl)-methanone;
(3-Hydroxy-6-aza-bicyclo[3.2.1]oct-6-yl)-(9-methyl-9H-carbazol-3-yl)-methanone
(9H-Carbazol-3-yl)-(3-hydroxy-6-aza-bicyclo[3.2.1]oct-6-yl)-methanone;
(6-Hydroxy-2-aza-bicyclo[2.2.1]hept-2-yl)-(9-methyl-9H-carbazol-3-yl)-methanone
(9H-Carbazol-3-yl)-(6-hydroxy-2-aza-bicyclo[2.2.1]hept-2-yl)-methanone;
9H-Carbazole-3-carboxylic acid (5-hydroxy-adamantan-2-yl)-amide;
9-Methyl-9H-carbazole-3-carboxylic acid (5-hydroxy-adamantan-2-yl)-methyl-amide;
9H-Carbazole-3-carboxylic acid (5-hydroxy-adamantan-2-yl)-methyl-amide
6-Chloro-9H-carbazole-3-carboxylic acid (trans 5-hydroxy-adamantan-2-yl)-amide;
(6-Chloro-9H-carbazol-3-yl)-(3-hydroxy-8-aza-bicyclo[3.2.1]oct-8-yl)-methanone;
6-Chloro-9H-carbazole-3-carboxylic acid (cis 5-hydroxy-adamantan-2-yl)-amide;
9H-Fluorene-3-carboxylic acid (3-hydroxy-adamantan-1-yl)-amide;
Dibenzofuran-2-yl-(3-hydroxy-8-aza-bicyclo[3.2.1]oct-8-yl)-methanone;
Dibenzofuran-2-yl-(3-hydroxy-8-aza-bicyclo[3.2.1]oct-8-yl)-methanone;
Dibenzofuran-2-carboxylic acid (5-hydroxy-adamantan-2-yl)-amide;
8-Chloro-dibenzofuran-2-carboxylic acid (5-hydroxy-adamantan-2-yl)-amide;
(8-Chloro-dibenzofuran-2-yl)-(3-hydroxy-8-aza-bicyclo[3.2.1]oct-8-yl)-methanone;
(3-Hydroxy-8-aza-bicyclo[3.2.1]oct-8-yl)-[8-(2-hydroxy-ethoxy)-dibenzofuran-2-yl]-methanone;
8-(2-Hydroxy-ethoxy)-dibenzofuran-2-carboxylic acid (5-hydroxy-adamantan-2-yl)-amide;
or a prodrug thereof, a salt thereof with a pharmaceutically acceptable acid or base, or any optical isomer or mixture of optical isomers, including a racemic mixture, or any tautomeric forms.

8. (canceled)

9. A method for the treatment, prevention and/or prophylaxis of any conditions, disorders and diseases that are influenced by intracellular glucocorticoid levels, the method comprising administering to a subject in need thereof an effective amount of a compound according to claim 1, or a salt thereof.

10. The method according to claim 9 which is an agent useful for the treatment, prevention and/or prophylaxis of conditions, disorders or diseases selected from the group consisting of the metabolic syndrome, insulin resistance, dyslipidemia, hypertension and obesity.

11-12. (canceled)

13. A pharmaceutical composition comprising a compound according to claim 1, or a salt thereof, and a pharmaceutically acceptable carrier or diluent.

14. A method for the treatment, prevention and/or prophylaxis of any conditions, disorders or diseases wherein a modulation or an inhibition of the activity of 11βHSD1 is beneficial, the method comprising administering to a subject in need thereof an effective amount of a compound according to claim 1, or a salt thereof.

15. The method according to claim 14 wherein the conditions, disorders or diseases are selected from the group consisting of the metabolic syndrome, insulin resistance, dyslipidemia, hypertension and obesity.
